(19) United States
(12) Patent Application Publication
(10) Pub. No.: US 2026/0071167 A1
(43) Pub. Date: Mar. 12, 2026

(54) ROTARY PERFUSION DEVICE FOR CULTURING BIOLOGICAL CELLS

(71) Applicant: The Trustees of Columbia University in the City of New York, New York, NY (US)

(72) Inventors: Keith Yeager, Springfield, NJ (US); Anjali Saqi, New York, NY (US); Kacey Ronaldson, New York, NY (US); Gordana Vunjak-Novakovic, New York, NY (US)

(21) Appl. No.: 19/386,490

(22) Filed: Nov. 12, 2025

Related U.S. Application Data
(63) Continuation of application No. PCT/US2024/029928, filed on May 17, 2024.
(60) Provisional application No. 63/503,363, filed on May 19, 2023.

(51) Int. Cl. C12M 1/00; C12M 1/12; C12M 3/06

(52) U.S. Cl. CPC C12M 29/10; C12M 23/34; C12M 23/50; C12M 25/04; C12M 27/14

(57) ABSTRACT

A culture vessel configured for culturing biological cells including a body being configured to be rotated about an axis of rotation, the body including one or more helical conduit configured to receive a fluid and extending along an exterior surface of the body, the body defining a reservoir configured to receive the fluid, and the body defining a first aperture and a second aperture each in communication with the helical conduit and the reservoir and configured for passage of the fluid, and the culture vessel further optionally including a support being received within the reservoir of the body, the support including a permeable membrane configured to accommodate biological cells, wherein the helical conduit of the body is configured to facilitate movement of the fluid between the first aperture and the second aperture of the body during rotation of the body.

### CROSS-REFERENCE TO RELATED APPLICATIONS

This application is a continuation of PCT International Application No. PCT/US2024/029928, filed May 17, 2024, which claims benefit of U.S. Provisional Application No. 63/503,363, filed May 19, 2023, the contents of each of which are hereby incorporated by reference.

### FIELD

The present disclosure relates to the field of biomedical engineering. More specifically, the present disclosure relates to devices, systems, and methods for culturing biological cells.

### GOVERNMENT FUNDING

This Invention was made with government support under CA013696, CA281356, CA285143, and HL170884 awarded by the National Institutes of Health. The government has certain rights in the invention.

### BACKGROUND

An organotypic bioreactor is a device and/or system used to grow and maintain functional three-dimensional cultures of biological cells, which make up biological tissues, in complex biological processes, such as tissue engineering, drug discovery, disease and toxicology research, and/or the like. Organotypic bioreactors recapitulate and/or simulate a physiological microenvironment of the tissues in vivo and allow the growth and maintenance of functional tissues in vitro. To this end, Organotypic bioreactors provide a system that enables control of nutrient delivery and cell culture in more physiologically relevant configurations. Organotypic bioreactors may also serve as platforms for studying tissue development, function, and response to various stimuli.

Traditional tissue culture systems, such as static, two-dimensional cultures, do not fully replicate an in vivo tissue microenvironment. In contrast, organotypic bioreactors provide a more physiologically relevant microenvironment, by incorporating elements such as mechanical forces, fluid flow, and cell-to-cell interactions.

One type of organotypic bioreactor is a perfusion-type organotypic bioreactor, which circulates a fluid to the cultured tissue. Perfusion-type organotypic bioreactors may also apply dynamic stimuli, such as nutrient and oxygen exchange, mechanical force (e.g. cyclic strain, pulsatile flow, steady flow, and/or the like), temperature and pH regulation, electrical and magnetic fields, and/or the like, which help to promote tissue maturation and remodeling by simulating a more realistic physiological microenvironment for the cultured tissue.

A typical perfusion-type organotypic bioreactor usually requires a complicated setup and operation, which requires skilled personnel and specialized equipment, making the typical perfusion-type organotypic bioreactor expensive and bulky. Complexity is exacerbated when scaling out (multiple in parallel) as multiple sets of tubing and pumps are required. Costs are also typically high due to the assembly, calibration and maintenance cost of multiple components both fluidic (tubing, reservoirs) and electromechanical (pumps, wiring, connectors).

Further, a typical pump-driven perfusion-type organotypic bioreactor has regions of high shear due to their cyclic nature and multi-component mechanical action and increased risk of contamination from multiple interacting/moving components which may detrimentally affect the growth and function of the cultured cells or tissue. Additionally, for example, generation of a high level of shear stress to the cultured cells or tissue, as a function of flow rate, viscosity, a geometry of a device used to culture the tissue, and/or the like, may result in cell and/or tissue damage or death, or otherwise alter cell behavior. The generation of heat from moving components, or particulate generation (e.g. spalling of tubing) from wear and tear of mechanically actuated components introduce unwanted elements into the culture system.

Accordingly, there is a need for an improved organotypic bioreactor capable of providing continuous perfusion and cellular expansion in an accessible, user-friendly, and small volume format, while also allowing for low-shear fluid mechanics and/or dynamic stimuli.

### SUMMARY

According to aspects of the present disclosure, a perfusion vessel configured for culturing biological cells is provided. The perfusion vessel includes a body extending between a first end and a second end and being configured to be rotated about an axis of rotation, the body including one or more helical conduits configured to receive a fluid and extending between the first end and the second end of the body along an exterior surface of the body, the body defining a reservoir configured to receive the fluid and extending between the first end and the second end of the body, and the body defining a first aperture and a second aperture each in communication with the helical conduit and the reservoir and configured for passage of the fluid, and the perfusion vessel further including a support extending between a first end and a second end, the support being received within the reservoir of the body, the support including a permeable membrane configured to accommodate biological cells, wherein the helical conduit of the body is configured to facilitate movement of the fluid between the first aperture and the second aperture of the body during rotation of the body.

According to aspects of the present disclosure, the helical conduit may be configured to facilitate movement of the fluid from the first aperture toward the second aperture during rotation of the body.

According to aspects of the present disclosure, the support may be configured to remain rotationally fixed relative to a longitudinal axis of the body during rotation of the body.

According to aspects of the present disclosure, the support may include a first wall and a second wall extending substantially perpendicular to the permeable membrane, the first wall may extend to a first height, the second wall may extend to a second height, and the first height of the first wall may be greater than the second height of the second wall.

According to aspects of the present disclosure, the first wall may be configured to contain a first column of the fluid when the fluid is received within the reservoir and the second wall may be configured to contain a second column of the fluid when the fluid is received within the reservoir.

According to aspects of the present disclosure, the support comprises side walls having a concave shape.

According to aspects of the present disclosure, the perfusion vessel may further include a container extending between a first end and a second end, the container may define a chamber extending between the first end and the second end of the container, and the chamber may be configured to receive the body.

According to aspects of the present disclosure, the container may define an opening at one or more of the first end and the second end of the container.

According to aspects of the present disclosure, the perfusion vessel may further include an endcap configured to releasably engage the opening of the container.

According to aspects of the present disclosure, one or more of the body and the endcap may define a flow port configured to direct the fluid into the reservoir.

According to aspects of the present disclosure, the flow port may include a spiroid channel configured to facilitate movement of the fluid during rotation of the body.

In the manner described and according to aspects illustrated herein, the perfusion vessel and method of use corresponding thereto are capable of providing continuous low-shear perfusion and nutrient delivery in an accessible, user-friendly, and small volume format.

Accordingly, the perfusion vessel and method of use corresponding thereto are capable of being used without expertise and/or infrastructure required for cell and/or tissue culture, while being capable of providing a more accurate representation of an in vivo tissue microenvironment, enabling optimized growth and maintenance of functional cell, tissues, or their products in vitro.

A perfusion vessel according to an exemplary embodiment of the present invention comprises: a body extending between a first end and a second end and being configured to be rotated about an axis of rotation, the body including one or more helical conduits configured to receive a fluid and extending between the first end and the second end of the body along an exterior surface of the body, the body defining a reservoir configured to receive the fluid and extending between the first end and the second end of the body, and the body defining a first aperture and a second aperture each in communication with the one or more helical conduits and the reservoir and configured for passage of the fluid; and optionally a support extending between a first end and a second end, the support being received within the reservoir of the body, the support including a plurality of wells configured to accommodate biological cells and a permeable membrane disposed below the plurality of wells; wherein the helical conduit of the body is configured to facilitate movement of the fluid between the first aperture and the second aperture of the body during rotation of the body.

In an exemplary embodiment, the perfusion vessel is configured for culturing biological cells.

In an exemplary embodiment, the support is configured to maintain orientation relative to a longitudinal axis of the body during rotation of the body.

In an exemplary embodiment, the support includes a first wall and a second wall extending substantially perpendicular to the permeable membrane, the first wall extends to a first height, the second wall extends to a second height, and the first height of the first wall is greater than the second height of the second wall.

In an exemplary embodiment, the first wall is configured to contain a first column of the fluid when the fluid is received within the reservoir and the second wall is configured to contain a second column of the fluid when the fluid is received within the reservoir.

In an exemplary embodiment, the support comprises side walls having a concave shape.

In an exemplary embodiment, the perfusion vessel further comprises a sheath disposed around the body, the sheath and the body forming a container that defines a chamber extending between a first end and a second end of the container.

In an exemplary embodiment, the container defines an opening at one or more of the first end and the second end of the container.

In an exemplary embodiment, the perfusion vessel further comprises an endcap configured to releasably engage the opening of the sheath.

In an exemplary embodiment, one or more of the body and the endcap defines a flow port configured to direct the fluid into the reservoir.

In an exemplary embodiment, the flow port comprises one or more sloped or curved channels configured to facilitate movement of the fluid during rotation of the body.

In an exemplary embodiment, the sheath is at least partially gas permeable.

According to an exemplary embodiment of the present invention, a perfusion vessel configured for culturing biological cells comprises: a body extending between a first end and a second end and being configured to be rotated about an axis of rotation, the body including one or more helical conduits configured to receive a fluid and extending between the first end and the second end of the body along an exterior surface of the body, the body defining a reservoir configured to receive the fluid and extending between the first end and the second end of the body, and the body defining a first aperture and a second aperture each in communication with the one or more helical conduits and the reservoir and configured for passage of the fluid, wherein the helical conduit of the body is configured to facilitate movement of the fluid between the first aperture and the second aperture of the body during rotation of the body.

In an exemplary embodiment, the vessel further comprises a support extending between a first end and a second end, the support being received within the reservoir of the body, the support including a plurality of wells configured to accommodate biological cells.

In an exemplary embodiment, the helical conduit is configured to facilitate movement of the fluid from the first aperture toward the second aperture during rotation of the body.

In an exemplary embodiment, the support is configured to remain orientation relative to a longitudinal axis of the body during rotation of the body.

In an exemplary embodiment, the support includes a first wall and a second wall extending substantially perpendicular to the permeable membrane, the first wall extends to a first height, the second wall extends to a second height, and the first height of the first wall is greater than the second height of the second wall.

In an exemplary embodiment, the first wall is configured to contain a first column of the fluid when the fluid is received within the reservoir and the second wall is configured to contain a second column of the fluid when the fluid is received within the reservoir.

In an exemplary embodiment, the support comprises side walls having a concave shape.

In an exemplary embodiment, the perfusion vessel further comprises a sheath disposed around the body, the sheath and the body forming a container that defines a chamber extending between the first end and the second end of the container.

In an exemplary embodiment, the container defines an opening at one or more of the first end and the second end of the container.

In an exemplary embodiment, the perfusion vessel further comprises an endcap configured to releasably engage the opening of the container.

In an exemplary embodiment, the sheath is at least partially gas permeable.

A support according to an exemplary embodiment comprises: a base extending between a first end and a second end; a platform extending across the base, the platform including one or more wells; and a permeable membrane extending across the platform, wherein the permeable membrane is configured such that cellular material can adhere to a surface of the membrane.

In an exemplary embodiment, the permeable membrane is configured to establish an interstitial flow resistance and/or interstitial flow rate of the culture medium perfused through the permeable membrane.

In an exemplary embodiment, the permeable membrane extends across the platform at a position beneath the platform.

In an exemplary embodiment, the support further includes a first wall and a second wall extending from a support floor, perpendicular or substantially perpendicular to the platform and the permeable membrane.

In an exemplary embodiment, the first wall extends from a position elevated and/or distanced from the floor of the base, at or adjacent to the platform and the second wall extends directly from the floor of the base.

In an exemplary embodiment, a cavity is defined between the first and second walls.

In an exemplary embodiment, the support comprises side walls having a concave shape.

A rotational media pump according to an exemplary embodiment of the present invention comprises: a main body comprising a first end portion and a second end portion, the main body configured to hold a fluid media; an inner tube disposed within the main body, the inner tube comprising one or more openings; and a helical structure disposed at at least one of the first or second end portions of the main body, the helical structure being in fluid communication with the inner tube, wherein, upon rotation of the main body, the helical structure rotates with the main body and transports the fluid media from an outer diameter of the main body into the inner tube, and the fluid media travels within the inner tube and exits from the one or more openings of the inner tube to once again arrive at the outer diameter of the main body so that the fluid media is continuously circulated within the main body.

In an exemplary embodiment, the main body comprises a sheath surrounding the inner tube and the helical structure.

In an exemplary embodiment, the main body further comprises one or more fluid connections.

In an exemplary embodiment, the main body further comprises one or more adapters each configured to attach to a vial.

A rotational media pump according to an exemplary embodiment of the present invention comprises: a main body comprising a first end portion and a second end portion, the main body configured to hold a fluid media; an inner tube disposed within the main body, the inner tube comprising one or more openings; and a spiral wound film disposed around the inner tube, the spiral wound film configured to support high density culture of adherent or suspension cells, wherein, upon rotation of the main body, the spiral wound film, rotates with the main body and transports the fluid media from an outer diameter of the main body into the inner tube, and the fluid media travels within the inner tube and exits from the one or more openings of the inner tube to once again arrive at the outer diameter of the main body so that the fluid media is continuously circulated within the main body.

In an exemplary embodiment, the main body comprises a sheath surrounding the inner tube and the spiral wound film.

In an exemplary embodiment, the main body further comprises one or more fluid connections.

In an exemplary embodiment, the main body further comprises one or more adapters each configured to attach to a vial.

In an exemplary embodiment, the rotational media pump further comprises a helical structure disposed at at least one of the first or second end portions of the main body, the helical structure being in fluid communication with the inner tube.

In an exemplary embodiment, the spiral wound film comprises a plurality of C-shaped openings configured to facilitate aggregation of cells in suspension up to a maximum size.

A multiwell plate system according to an exemplary embodiment of the present invention comprises: one or more multiwell plates; one or more rotational media pumps disposed adjacent to the one or more multiwell plates; and one or more connection elements disposed between the one or more multiwell plates and the one or more rotational media pumps, wherein, upon rotation of the one or more rotational media pumps, fluid media is pumped through the one or more connections to the one or more multiwell plates, the fluid media travels across the multiwell plates, and the fluid media enters the one or more connections to once again enter the one or more rotational media pumps so that the fluid media is continuously circulated across the multiwell plates.

In an exemplary embodiment, at least one of the one or more rotational media pumps comprises: a main body comprising a first end portion and a second end portion, the main body configured to hold a fluid media; an inner tube disposed within the main body, the inner tube comprising one or more openings; and a helical structure disposed at at least one of the first or second end portions of the main body, the helical structure being in fluid communication with the inner tube, wherein, upon rotation of the main body, the helical structure rotates with the main body and transports the fluid media from an outer diameter of the main body into the inner tube, and the fluid media travels within the inner tube and exits from the one or more openings of the inner tube to once again arrive at the outer diameter of the main body so that the fluid media is continuously circulated within the main body.

In an exemplary embodiment, at least one of the one or more rotational media pumps comprises: a main body comprising a first end portion and a second end portion, the main body configured to hold a fluid media; an inner tube disposed within the main body, the inner tube comprising one or more openings; and a spiral wound film disposed around the inner tube, wherein, upon rotation of the main body, the spiral wound film rotates with the main body and transports the fluid media from an outer diameter of the main body into the inner tube, and the fluid media travels within the inner tube and exits from the one or more openings of the inner tube to once again arrive at the outer diameter of the main body so that the fluid media is continuously circulated within the main body, or upon rotation of the main body, the spiral wound film rotates with the main body and transports the fluid media from the inner tube to an outer diameter of the main body, and the fluid media travels within the main body and enters the one or more openings of the inner tube so that the fluid media is continuously circulated within the main body.

In an exemplary embodiment, the multiwell plates are cell culture plates.

In an exemplary embodiment, the multiwell plates are arranged on a slope so that the fluid media travels across the multiwell plates due to force of gravity.

A system according to an exemplary embodiment of the present invention comprises a series of parallel roller bars, each having a longitudinal axis and rotatable therearound, having disposed longitudinally thereon one or more of any of the previously-described devices containing fluid therein, wherein, when said parallel roller bars rotate, fluid flow is effected in the one or more devices disposed longitudinally thereon.

In an exemplary embodiment, the fluid comprises a cell culture media.

A system according to an exemplary embodiment of the present invention comprises a carousel configured to rotate about an axis of rotation and hold one or more of any of the previously-described devices.

A bioreactor system according to an exemplary embodiment of the present invention comprises one or more of any of the previously-described devices or systems, and the system is configured to have size specified mesh sieve/screens along a media flow route to enable splitting or passaging of cell aggregates larger than the specified size of mesh holes (range 1um-600um) enabling continuous culture of suspended aggregates of a defined size.

A bioreactor system according to an exemplary embodiment of the present invention comprises one or more of any of the previously-described devices or systems, and the system is configured to selectively activate cells via coatings of inner components (including but not limited to the support) with biomimetic signals such as but not limited to cytokines or antigen presenting cells (natural or artificial), increase cell activation via culture in high density discrete organotypic niches and engineer cells (via delivery of virus through transduction or transfection or other approaches such as CRISPR-Cas9).

In an exemplary embodiment, the surface coatings include hydrophobic versus hydrophilic options, molecules including proteins, peptides, antibodies, enzymes, and other target-specific molecules aimed at inducing an expected cellular responses, cell based coatings or “feeder-cells” (irradiated or non irradiated), antigen presenting cells (naturally derived or engineered), biodegradable/time release coatings for slow addition of compounds of interest, and hydrogels.

A bioreactor system according to an exemplary embodiment of the present invention comprises one or more of any of the previously-described devices or systems, and the system is configured to either maintain the initial state of cells or enhance the differentiation and/or maturation of cells via external factors including but not limited to temporal delivery of chemical factors, shear stress, aggregate size, and antigen activation.

A bioreactor system according to an exemplary embodiment of the present invention comprises one or more of any of the previously-described devices or systems, and the system is configured to support the culture of 3D engineered tissues and/or tissue explants.

A bioreactor system according to an exemplary embodiment of the present invention comprises one or more of any of the previously-described devices or systems, and the system is configured to selectively filter substances, including but not limited to wastewater treatment or enrichment of cell cultures.

A bioreactor system according to an exemplary embodiment of the present invention comprises one or more of any of the previously-described devices or systems, and the system is configured for the expansion and culture of mammalian cells, including human and mouse cells, in 2D, 3D, or on microcarriers.

A bioreactor system according to an exemplary embodiment of the present invention comprises one or more of any of the previously-described devices or systems, and the system is configured for the expansion and culture of non-mammalian cells, including insect and other types of cells, in 2D, 3D, or on microcarriers.

A bioreactor system according to an exemplary embodiment of the present invention comprises one or more of any of the previously-described devices or systems, and the system is configured for the expansion and culture of other cell types, including plant, bacteria, yeast, and fungus cells, in 2D, 3D, or on microcarriers.

A bioreactor system according to an exemplary embodiment of the present invention comprises one or more of any of the previously-described devices or systems, and the system is configured for culturing single or multi-cell suspensions, aggregate suspensions, organoids, adherent cultures, and 3D tissues.

A bioreactor system according to an exemplary embodiment of the present invention comprises one or more of any of the previously-described devices or systems, and the system is configured for the production of cell based therapeutics and cell-produced factors including biologics, proteins, antibodies, exosomes, cytokines, and mitochondria.

A bioreactor system according to an exemplary embodiment of the present invention comprises one or more of any of the previously-described devices or systems, and the system is configured for applications in therapeutic production, disease modeling, screening, bio-banking, and diagnostics.

A bioreactor system according to an exemplary embodiment of the present invention comprises one or more of any of the previously-described devices or systems, and the system is configured for vaccine production.

A bioreactor system according to an exemplary embodiment of the present invention comprises one or more of any of the previously-described devices or systems, and the system is configured for precision fermentation.

A bioreactor system according to an exemplary embodiment of the present invention comprises one or more of any of the previously-described devices or systems, and the system is configured for microbial bioproduction.

A bioreactor system according to an exemplary embodiment of the present invention comprises one or more of any of the previously-described devices or systems, and the system is configured for stem cell maintenance and differentiation.

A bioreactor system according to an exemplary embodiment of the present invention comprises one or more of any of the previously-described devices or systems, and the system is configured for transduction processes.

A bioreactor system according to an exemplary embodiment of the present invention comprises one or more of any of the previously-described devices or systems, and the system is configured for the local preparation of prebiotics, probiotics, and postbiotics for consumption or therapy, such as gut, skin, and stool microbiome applications, to eliminate the need for the addition of preservatives.

A bioreactor system according to an exemplary embodiment of the present invention comprises one or more of any of the previously-described devices or systems, and the system is configured for the culture of bacterial cells to produce biologics.

A bioreactor system according to an exemplary embodiment of the present invention comprises one or more of any of the previously-described devices or systems, and the system is configured for the culture of Chinese Hamster Ovary (CHO) cells to produce biologics.

A bioreactor system according to an exemplary embodiment of the present invention comprises one or more of any of the previously-described devices or systems, and the system is configured for the culture of Human Embryonic Kidney (HEK) cells to produce biologics.

A bioreactor system according to an exemplary embodiment of the present invention comprises one or more of any of the previously-described devices or systems, and the system is configured for the production of therapeutic proteins.

A bioreactor system according to an exemplary embodiment of the present invention comprises one or more of any of the previously-described devices or systems, and the system is configured for the production of peptides.

A bioreactor system according to an exemplary embodiment of the present invention comprises one or more of any of the previously-described devices or systems, and the system is configured for the production of cytokines.

A bioreactor system according to an exemplary embodiment of the present invention comprises one or more of any of the previously-described devices or systems, and the system is configured for the production of exosomes.

A bioreactor system according to an exemplary embodiment of the present invention comprises one or more of any of the previously-described devices or systems, and the system is configured for the culture of engineered cells to produce biologics.

In an exemplary embodiment, the culture medium is adapted for the production of bioengineered tissues.

A bioreactor system according to an exemplary embodiment of the present invention comprises one or more of any of the previously-described devices or systems, and the system is configured to operate under both batch and continuous culture modes for the production of biologics.

A bioreactor system according to an exemplary embodiment of the present invention comprises one or more of any of the previously-described devices or systems, and the system includes an adaptable culture platform for transitioning between different cell types, including natural and engineered cells, bacterial cells, CHO cells, and HEK cells, without the need for significant reconfiguration.

A bioreactor system according to an exemplary embodiment of the present invention comprises one or more of any of the previously-described devices or systems, and the system includes specialized growth media and supplements tailored for specific cell types and production goals, such as media optimized for high-yield production of therapeutic proteins, peptides, cytokines, exosomes, and other biologics.

A bioreactor system according to an exemplary embodiment of the present invention comprises one or more of any of the previously-described devices or systems, and the system is configured for the accelerated aging of spirits, including the production of probiotic alcohol drinks.

A bioreactor system according to an exemplary embodiment of the present invention comprises one or more of any of the previously-described devices or systems, and the system includes a cork-based insert to enhance the aging process of spirits.

A bioreactor system according to an exemplary embodiment of the present invention comprises one or more of any of the previously-described devices or systems, and the system is configured for batch or continuous culture of bacteria and yeast-based drinks.

A bioreactor system according to an exemplary embodiment of the present invention comprises one or more of any of the previously-described devices or systems, and the system includes a mixing mechanism to ensure homogeneous culture conditions.

A bioreactor system according to an exemplary embodiment of the present invention comprises one or more of any of the previously-described devices or systems, and the system includes temperature control capabilities for heating and cooling fluids, featuring high conductive transfer areas to maintain optimal culture conditions.

A bioreactor system according to an exemplary embodiment of the present invention comprises one or more of any of the previously-described devices or systems, and the system is configured for the removal of contaminants from solutions, including wastewater treatment applications.

### BRIEF DESCRIPTION OF THE DRAWINGS

Aspects of an embodiment will be described with reference to the drawings, where like numerals reflect like elements:

FIG. 1 is a partially transparent side view of a perfusion vessel according to aspects of the present disclosure;

FIG. 2 is a side exploded view of the perfusion vessel according to FIG. 1;

FIG. 3 is a side cross-sectional view of the perfusion vessel according to FIG. 1;

FIG. 4 is a side view of a body of the perfusion vessel according to FIG. 1;

FIG. 5 is a side cross-sectional view of the body of the perfusion vessel according to FIG. 4;

FIG. 6 is a rear view of the body of the perfusion vessel according to FIG. 1;

FIG. 7 is a top view of a support of the perfusion vessel according to FIG. 1;

FIG. 8 is a partial enlarged front cross-sectional view of the support of the perfusion vessel according to FIG. 7;

FIG. 9 is a top view of an alternative configuration of a support of the perfusion vessel according to FIG. 1;

FIG. 10 is a partial enlarged front cross-sectional view of the support of the perfusion vessel according to FIG. 9;

FIG. 11 is a side cross-sectional view of the support of the perfusion vessel according to FIG. 9.

FIGS. 12A is a side view of a rotational media pump according to an exemplary embodiment of the present invention;

FIGS. 12B-12D are side views of helical channels according to exemplary embodiments of the present invention;

FIG. 13A is a perspective view of a rotational media pump according to an exemplary embodiment of the present invention;

FIGS. 13B-13E are cross sectional views of the rotational media pump of FIG. 13A showing operation of the rotational media pump according to an exemplary embodiment of the present invention;

FIGS. 14A is a front view of an insert according to an exemplary embodiment of the present invention;

FIG. 14B is a cross sectional view of a rotational media pump according to an exemplary embodiment of the present invention;

FIG. 15 is a cross-sectional view of an insert according to an exemplary embodiment of the present invention;

FIG. 16 is a photograph showing a screen according to an exemplary embodiment of the present invention;

FIGS. 17A and 17B are perspective views of a rotational media pump according to an exemplary embodiment of the present invention;

FIG. 17C is a cross-sectional view of the rotational media pump of FIGS. 17A and 17B according to an exemplary embodiment of the present invention;

FIG. 17D is a perspective view of the rotational media pump of FIGS. 17A and 17B according to an exemplary embodiment of the present invention;

FIG. 17E is a perspective view of a spiral wound film according to an exemplary embodiment of the present invention;

FIG. 17F is a detailed view of a surface of a spiral wound film according to an exemplary embodiment of the present invention;

FIG. 18A is a perspective view of a multiwell plate system according to an exemplary embodiment of the present invention;

FIG. 18B is a top view of the multiwell plate system of FIG. 18A; and

FIG. 18C is a cross-sectional view of the multiwell plate system of FIG. 18A.

### DETAILED DESCRIPTION

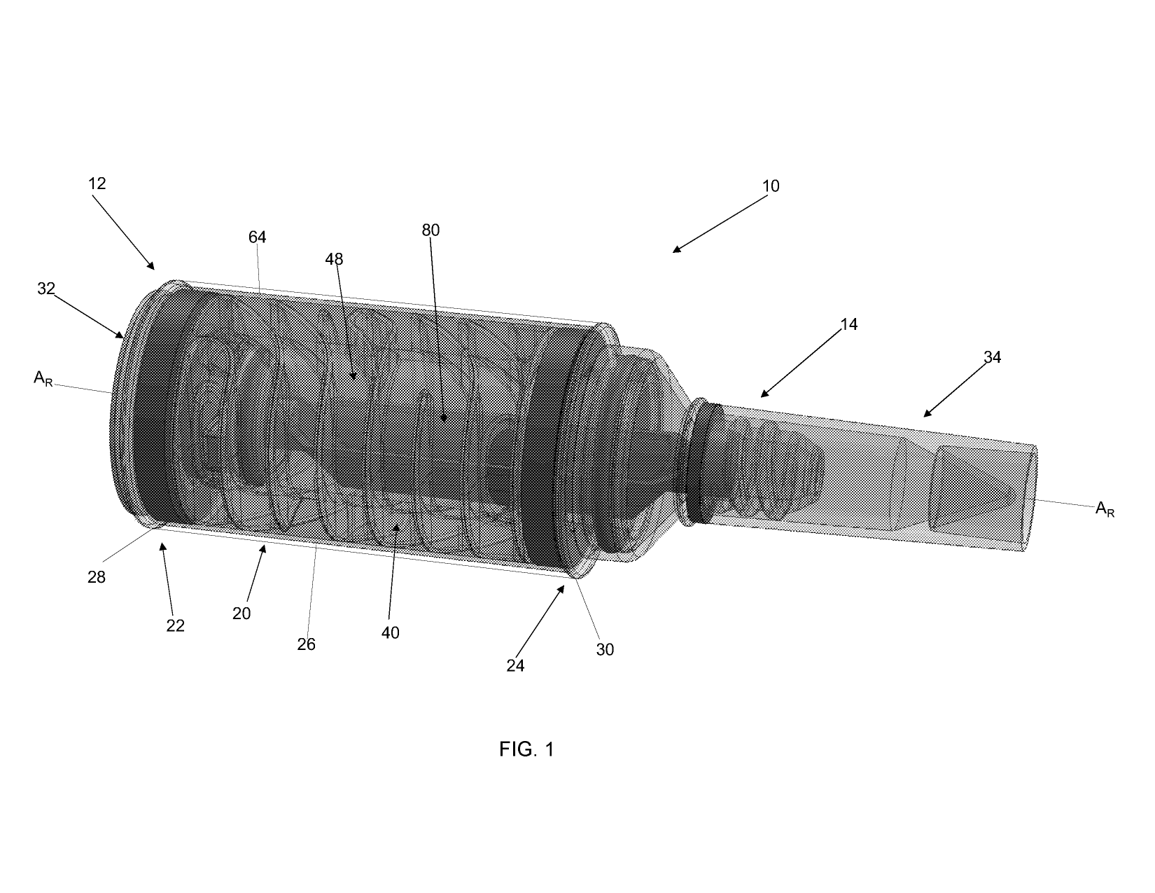

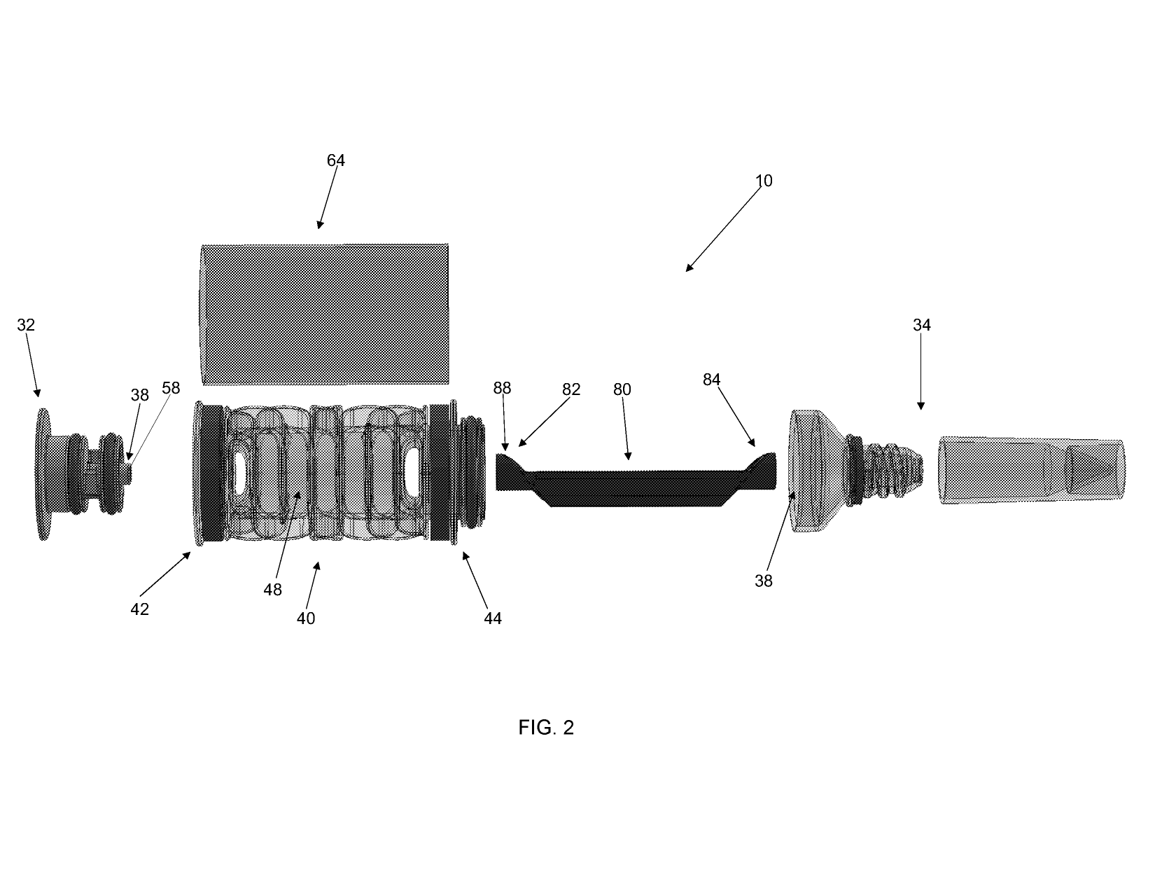

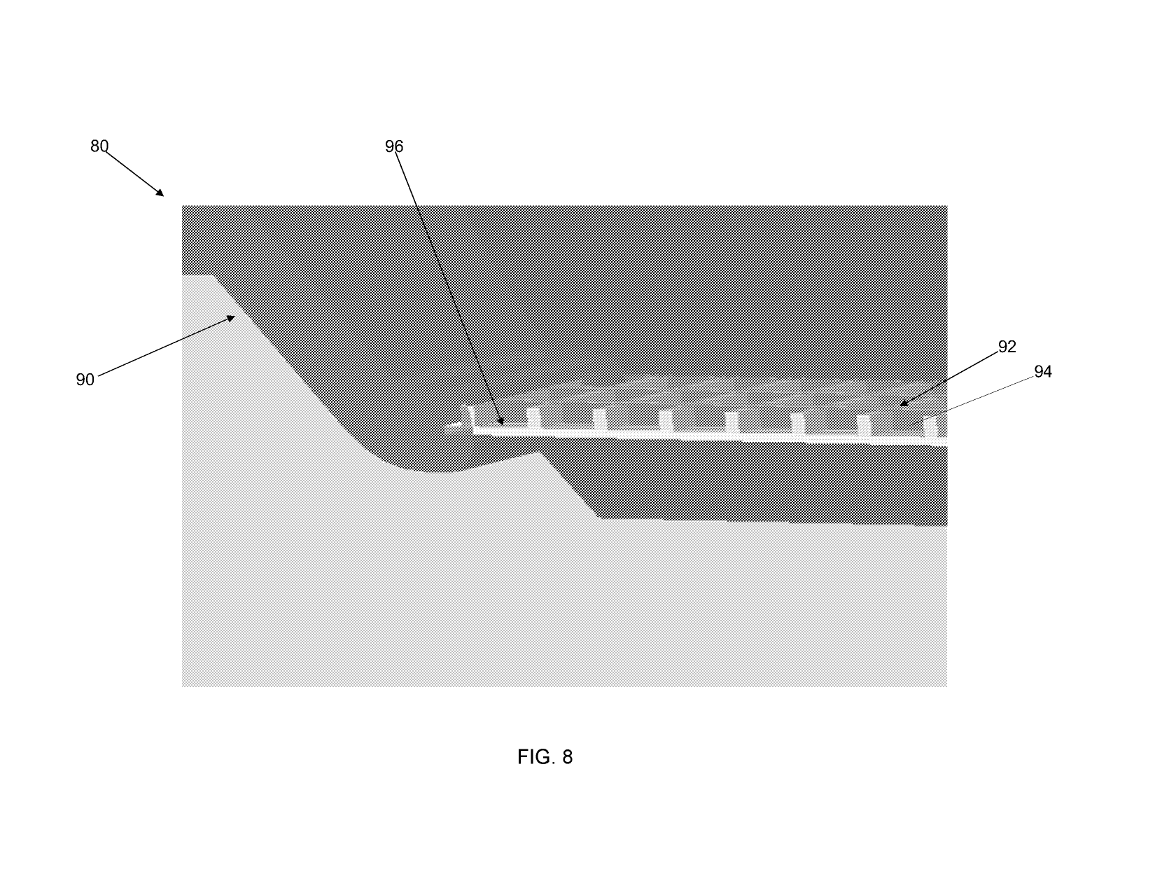

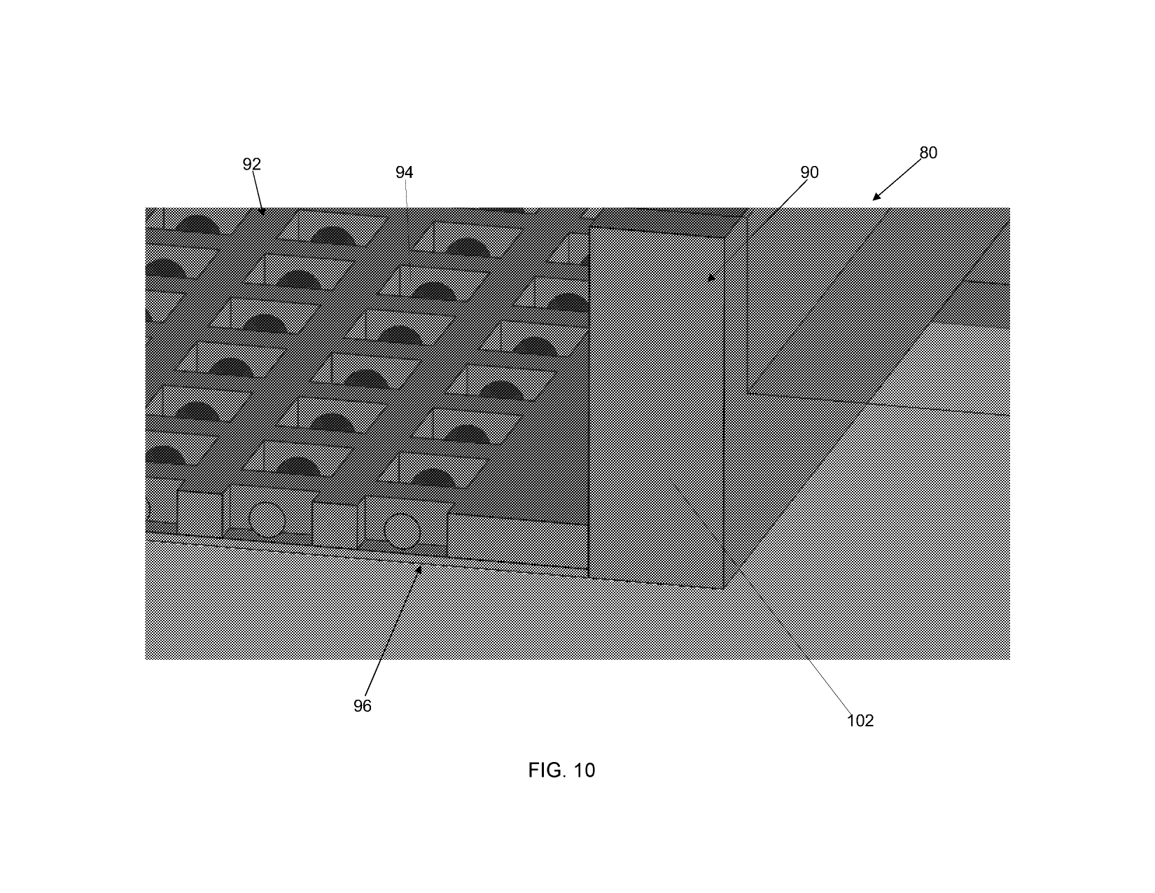

An embodiment of a perfusion vessel according to aspects of the present disclosure will now be described with reference to FIGS. 1-11. Like numerals represent like parts, and the perfusion vessel will generally be referred to by the reference numeral 10. Although the perfusion vessel 10 is described with reference to specific examples, it should be understood that modifications and changes may be made to these examples without going beyond the general scope as defined by the claims. In particular, individual characteristics of the various embodiments shown and/or mentioned herein may be combined in additional embodiments. Consequently, the description and the drawings should be considered in a sense that is illustrative rather than restrictive. The Figures, which are not necessarily to scale, depict illustrative aspects and are not intended to limit the scope of the present disclosure. The illustrative aspects depicted are intended only as exemplary.

The term “exemplary” is used in the sense of “example,” rather than “ideal.” While aspects of the present disclosure are amenable to various modifications and alternative forms, specifics thereof have been shown by way of example in the drawings and will be described in detail. It should be understood, however, that the intention is not to limit aspects of the present disclosure to the particular embodiment(s) described. On the contrary, the intention of the present disclosure is to cover all modifications, equivalents, and alternatives falling within the scope of the present disclosure.

Various materials, methods of construction and methods of fastening will be discussed in the context of the disclosed embodiment(s). Those skilled in the art will recognize known substitutes for the materials, construction methods, and fastening methods, all of which are contemplated as compatible with the disclosed embodiment(s) and are intended to be encompassed by the appended claims.

As used in the present disclosure and the appended claims, the singular forms “a,” “an,” and “the” include plural referents unless the content clearly dictates otherwise. As used in the present disclosure and the appended claims, the term “or” is generally employed in its sense including “and/or” unless the content clearly dictates otherwise.

Throughout the description, including the claims, the terms "comprising a,” “including a,” and “having a” should be understood as being synonymous with "comprising one or more," “including one or more,” and “having one or more” unless otherwise stated. In addition, any range set forth in the description, including the claims should be understood as including its end value(s) unless otherwise stated. Specific values for described elements should be understood to be within accepted manufacturing or industry tolerances known to one of skill in the art, and any use of the terms "substantially," "approximately," and “generally” should be understood to mean falling within such accepted tolerances.

When an element or feature is referred to herein as being “on,” “engaged to,” “connected to,” or “coupled to” another element or feature, it may be directly on, engaged, connected, or coupled to the other element or feature, or intervening elements or features may be present. In contrast, when an element or feature is referred to as being “directly on,” “directly engaged to,” “directly connected to,” or “directly coupled to” another element or feature, there may be no intervening elements or features present. Other words used to describe the relationship between elements or features should be interpreted in a like fashion (e.g., “between” versus “directly between,” “adjacent” versus “directly adjacent,” etc.).

Spatially relative terms, such as “top,” “bottom,” “middle,” “inner,” “outer,” “beneath,” “below,” “lower,” “above,” “upper,” and/or the like, may be used herein for ease of description to describe one element or feature's relationship to another element(s) or feature(s) as illustrated in the drawings. Spatially relative terms may be intended to encompass different orientations of a device in use or operation in addition to the orientation depicted in the drawings. For example, if the device in the drawings is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Although the terms “first,” “second,” etc. may be used herein to describe various elements, components, regions, layers, sections, and/or parameters, these elements, components, regions, layers, sections, and/or parameters should not be limited by these terms. These terms are only used to distinguish one element, component, region, layer, or section from another region, layer, or section. Thus, a first element, component, region, layer, or section discussed herein could be termed a second element, component, region, layer, or section without departing from the teachings of the present disclosure.

For the purposes of the present disclosure, the term “perfusion vessel” encompasses culture vessels, reaction vessels, mixing vessels and heat transfer vessels.

Also, for the purposes of the present disclosure, the term “media pump” may be used interchangeably with “perfusion vessel” or may refer to a structure that forms a part of a perfusion vessel. As shown in FIGS. 1-3, a perfusion vessel 10 configured for culturing biological, engineered, and/or artificial cells and/or tissue, or the products generated by such (hereafter, collectively referred to as “the cultured tissue”) is disclosed. In examples, the perfusion vessel 10 extends between a first end 12 and a second end 14 and is configured to be rotated about an axis of rotation A_{R}. It is contemplated that other components of the perfusion vessel 10 are also configured to rotate about the axis of rotation A_{R} of the perfusion vessel 10 and, thus, each corresponding component may be understood as including an axis of rotation that is common to and/or the same as the axis of rotation A_{R} of the perfusion vessel 10. By rotating the perfusion vessel 10, a culture medium is perfused throughout the perfusion vessel 10 and, thus, to the cultured tissue. It is contemplated that the culture medium may be understood to be a fluid or fluid-like composition supplemented with various nutrients, growth factors, and/or the like to promote cellular growth, differentiation, and survival.

It is contemplated that the perfusion vessel 10 may be rotated with known devices, such as a roller apparatus (not shown) including rotating cylinders configured to impart a rolling motion, or a tube rotator in which the perfusion vessel 10 is secured in a rotor at various locations along its radius/area and the rotor is rotated. In such an example, the perfusion vessel 10 is placed into the rolling apparatus, atop the rotating cylinders, and rotated at a controlled speed. In examples, the rotating speed may be within a range of 0.01 to 200 revolutions per minute (RPM), depending on a type of the cultured tissue and/or desired culture conditions. In certain examples, this speed may be lowered to .01 RPM or raised towards 200 RPM. The imparted rolling motion provides the cultured tissue with a gentle and uniform agitation, thereby enhancing an exchange of nutrients, oxygen, and waste products between the cultured tissue and the culture medium.

Referring to FIGS. 1-3, the perfusion vessel 10 includes a sheath 64 configured to surround, protect, and/or separate aspects of the perfusion vessel 10 from an external environment, a body 40 configured to be received within the sheath 64 and to perfuse the culture medium within the perfusion vessel 10, and a support 80 configured to be received within the body 40 and to hold the cultured tissue. The combination of the body 40 and sheath 64 shall be defined as the container 20. It is contemplated that an environment external to the perfusion vessel 10 may be any area and/or surrounding environment external to and/or outside of the container 20 of the perfusion vessel 10. It is contemplated that an interior of the perfusion vessel 10 may be any area internal to and/or inside of the container 20 of the perfusion vessel 10.

Referring to FIG. 1, the container 20 extends axially between a first end 22 and a second end 24 and is configured to be rotated about the axis of rotation A_{R}. The container 20 may be tubular or substantially tubular in shape. The container 20 defines an interior chamber 26 extending between the first end 22 and the second end 24 of the container 20. The chamber 26 is configured to receive the support 80. The container 20 may define an opening (also referred to herein as a “first opening”) 28 at the first end 22 of the container 20. Additionally or alternatively, the container 20 may define a first opening 28 at the first end 22 of the container 20 and a second opening 30 at the second end 24 of the container 20; however, the container 20 will be described with reference to “the opening 28” unless reference to the first opening 28 and the second opening 30 is otherwise necessary. The opening 28 may be in communication with the chamber 26. The opening 28 may be configured to receive an endcap 32. Additionally or alternatively, the opening 28 may be configured to mate with the endcap 32. Additionally or alternatively, the opening 28 may be configured to mate with an adapter 34 configured to adapt the perfusion vessel 10 for various research operations and/or to connect the perfusion vessel 10 to various research equipment (not shown), such as, for example, other tubes or connectors whether barbed, luer, threaded, glued, etc. Additionally or alternatively, the endcap 32 and/or the adaptor 34 may be integrally formed as and/or considered part of one or more of the container 20 and the body 40 so as to form a unitary structure. The opening 28 and the endcap 32 may include complimentary fastening mechanisms, such as a threaded arrangement, a bayonet connection, an interference-fit arrangement, and/or the like. Additionally or alternatively, the endcap 32 may be configured to mate with the body 40 and, thus, the body 40 and the endcap 32 may include complimentary fastening mechanisms that are the same or substantially similar to the complimentary fastening mechanisms described with respect to the opening 28 of the container 20 and the endcap 32. Further, one or more of the container 20, the body 40, the endcap 32, and the adapter 34 may include an elastomeric seal 36 configured to seal an interface between one or more of the body 40 and the container 20, the endcap 32 and the body 40 and/or the container 20, and the adapter 34 and the body 40 and/or the container 20. In examples, the endcap 32 may include a port to allow for ease of access to the interior of the perfusion vessel 10. In this manner, the container 20 is configured to surround, protect, and/or separate aspects of the perfusion vessel 10 from the external environment.

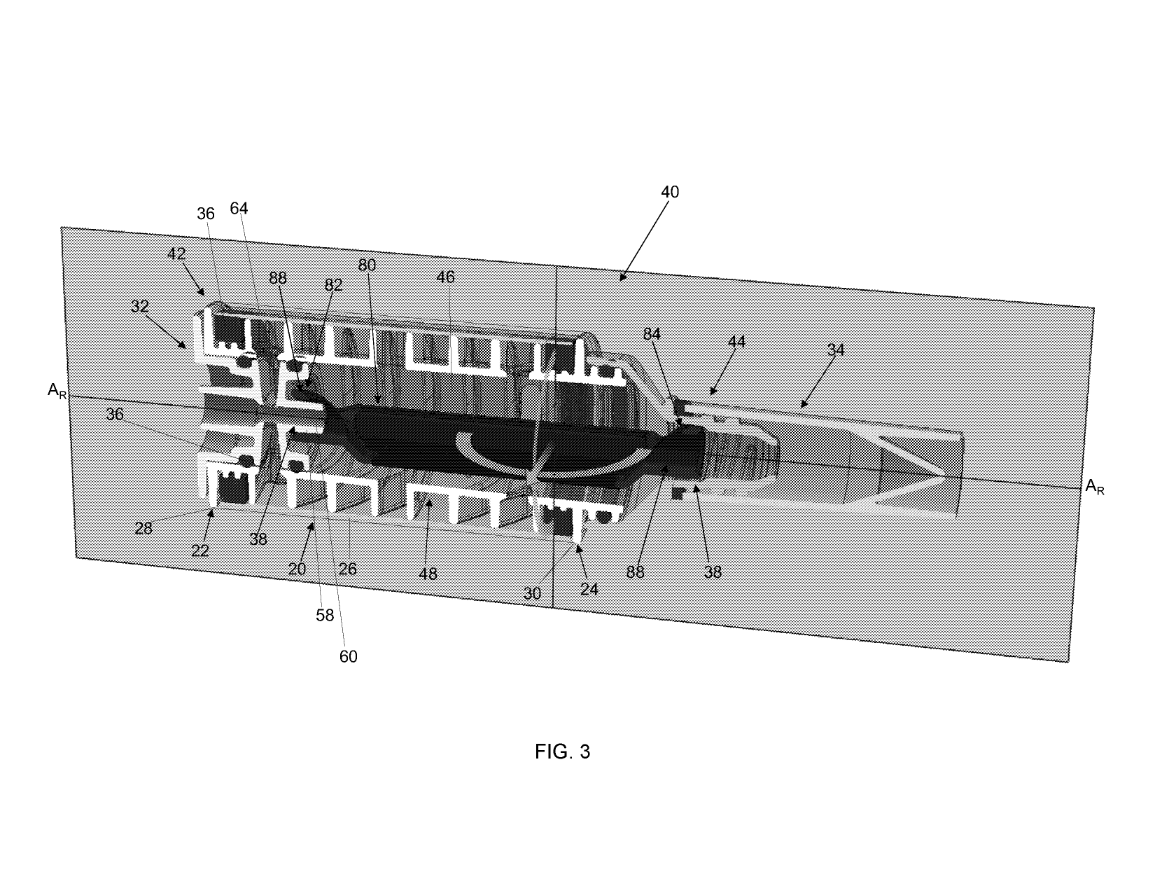

As shown in FIGS. 3-5, the body 40 extends axially between a first end 42 and a second end 44 and is configured to be rotated about the axis of rotation A_{R}. It is contemplated that the first end 42 of the body 40 may be positioned at or adjacent to the first end 22 of the container 20 and the second end 44 of the body 40 may be positioned at or adjacent to the second end 24 of the container 20 when the body 40 is received by the container 20. Additionally or alternatively, it is contemplated that the container 20 may be integrally formed as and/or considered part of the body 40 so as to form a unitary structure. The body 40 may be tubular or substantially tubular in shape. The body 40 defines a reservoir 46 extending between the first end 42 and the second end 44 of the body 40. The reservoir 46 is configured to receive the support 80. Additionally or alternatively, the body 40 is configured to bear the support 80 within the reservoir 46. To this end, one or more of the body 40 and the endcap 32 includes a first engagement member 38 and the support 80 includes a second engagement member 88 and the first engagement member 38 and the second engagement member 88 are configured to engage with each other. The first engagement member 38 may be included at the first end 42 and the second end 44 of the body 40 to engage the support 80 at opposing ends of the support 80. Additionally or alternatively, the first engagement member 38 may be included on an end of the endcap 32. The first engagement member 38 of the body 40 and/or the endcap 32 and the second engagement member 88 of the support 80 may be configured to engage each other such that the support 80 is configured to face in a fixed direction during rotation of the body 40. To this end, the first engagement member 38 and the second engagement member 88 may be in the form of a hinge joint, a ball and socket, and/or the like.

Referring to FIGS. 3-5, the body 40 is configured to perfuse the culture medium throughout the perfusion vessel 10. In particular, the body 40 is configured to perfuse the culture medium through the reservoir 46 of the body 40. To this end, the body 40 includes one or more helical conduits (also referred to herein as “the first helical conduit”) 48 extending between the first end 42 and the second end 44 of the body 40. The helical conduit 48 extends axially between a first end 50 and a second end 52. In examples, the body 40 may include a plurality of helical conduits 48, thereby further reducing pulsatile flow of the culture medium and/or providing an increasingly continuous flow of the culture medium; however, the body 40 will be described with reference to “the helical conduit 48,” unless reference to a plurality of helical conduits 48 is otherwise necessary. The helical conduit 48 is oriented radially about the body 40, along an exterior surface of the body 40.

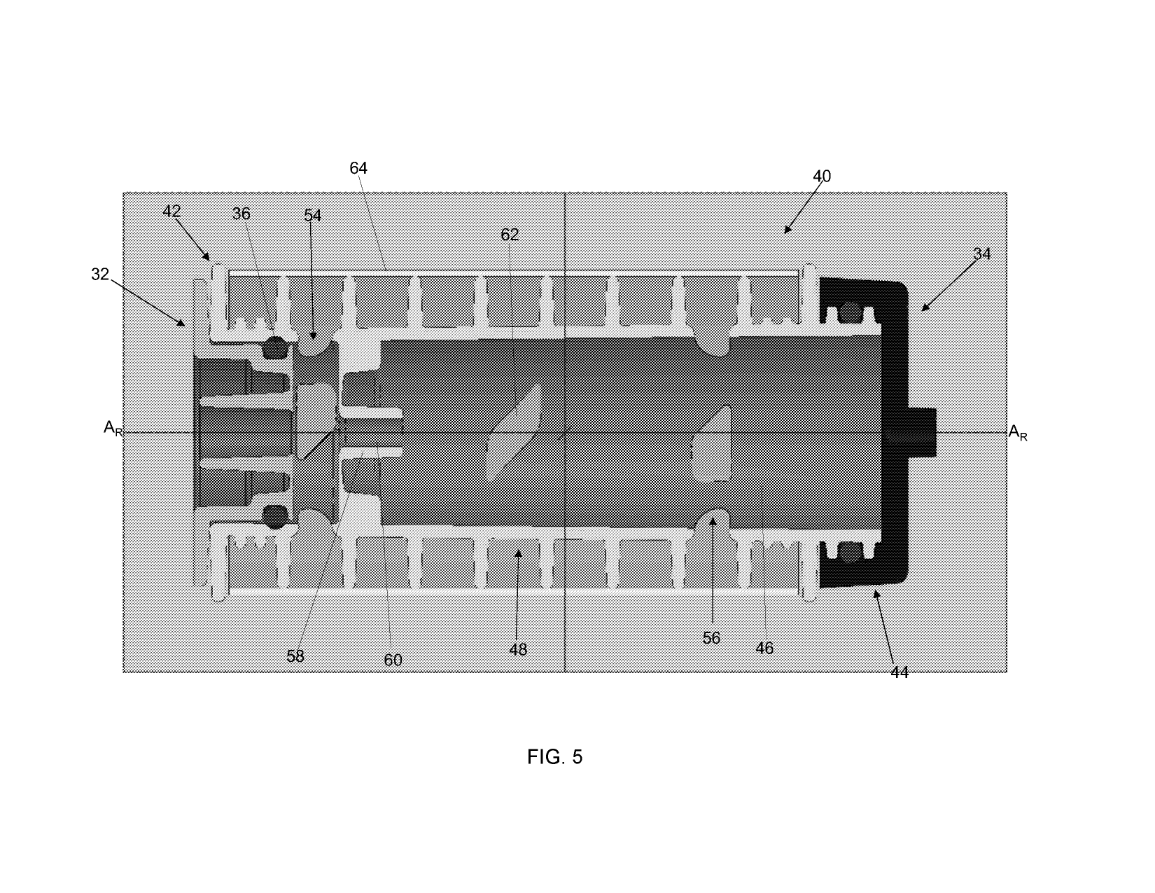

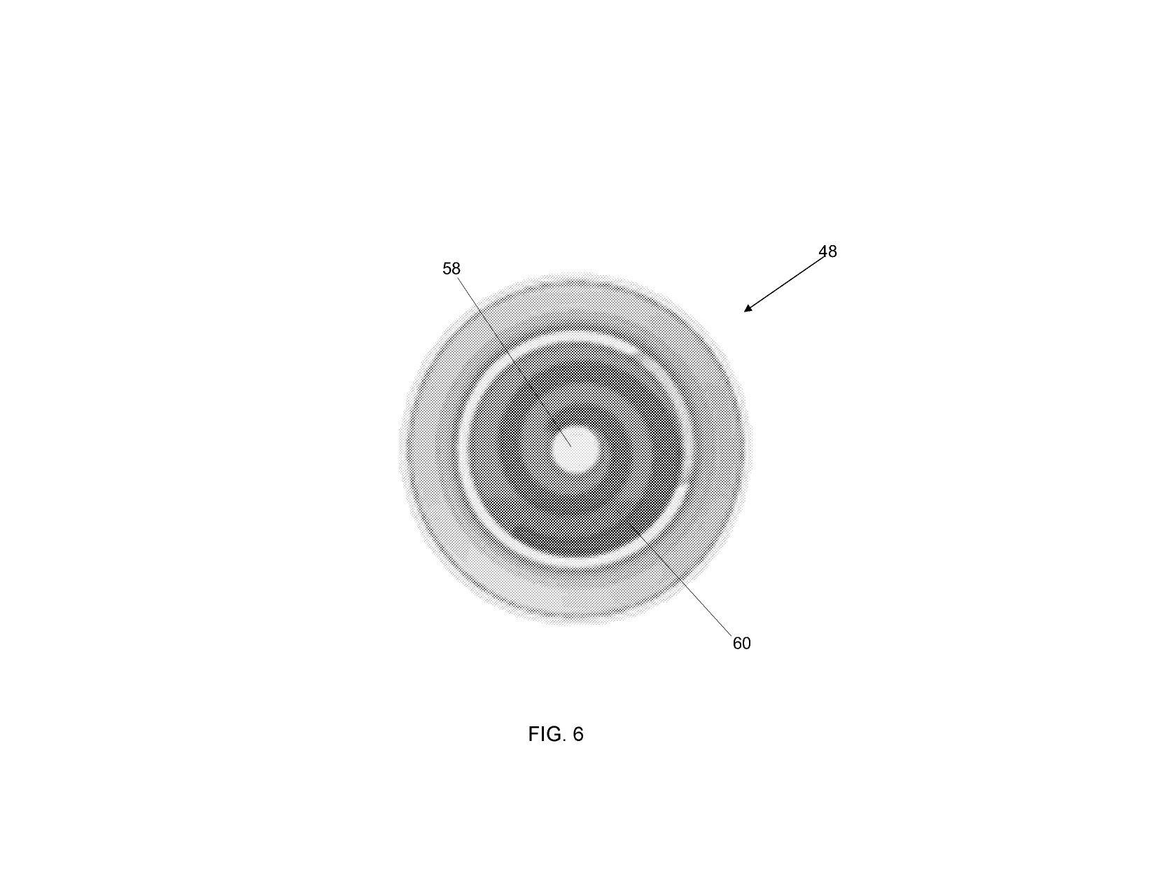

The body 40 defines at least a first aperture (also may be referred to herein as an “inlet”) 54 and a second aperture (also may be referred to herein as an “outlet”) 56, each in communication with the helical conduit 48 and the reservoir 46. In examples, the first aperture 54 may be configured to allow the culture medium to enter the reservoir 46 and the second aperture 56 may be configured to allow the culture medium to exit the reservoir 46 when the body 40 is rotated in a first direction. Additionally or alternatively, the first aperture 54 may be configured to allow the culture medium to exit the reservoir 46 and the second aperture 56 may be configured to allow the culture medium to enter the reservoir 46 when the body 40 is rotated in a second direction opposite the first direction of rotation. However, the first aperture 54 and the second aperture 56 will be described with respect to rotation of the body 40 in the first direction, unless rotation of the body 40 in the second direction is otherwise necessary. In examples, the first aperture 54 is defined at or adjacent to the first end 42 of the body 40 and the second aperture 56 is defined at or adjacent to the second end 44 of the body 40. Additionally or alternatively, a flow port 58 may be included at or adjacent to the first end 42 of the body 40. In particular, the flow port 58 may be positioned between the first aperture 54 and the reservoir 46, such that the first aperture 54 is separated from the reservoir 46 and/or is in indirect communication with the reservoir 46. Alternatively, the flow port 58 may be included at or adjacent to the second end of the body 40. As such, the flow port 58 may be positioned between the second aperture 56 and the reservoir 46, such that the second aperture 56 is separated from the reservoir 46 and/or is in indirect communication with the reservoir 46. The flow port 58 may be included on the body 40 (see FIG. 5). Additionally or alternatively, the flow port 58 may be included on the endcap 32 (see FIG. 3). The flow port 58 may be configured to increase control over the flow of the culture medium within the perfusion vessel 10. The flow port 58 may include a channel 60 extending into the reservoir 46. The channel 60 of the flow port 58 may be in the form of a spiroid (see FIG. 6), so as to function as a scoop pump and/or centrifugal pump, utilizing a Venturi-type effect to move the culture medium from the exterior of the body 40 into the reservoir 46. It is contemplated that the flow port 58 may also be configured to function as an adaptor for connecting tissue-specific inserts (not shown) configured to be compatible with the perfusion vessel 10 (e.g. an insert configured for culturing bone tissue, cardiac tissue, pulmonary tissue, and/or the like, respectively).

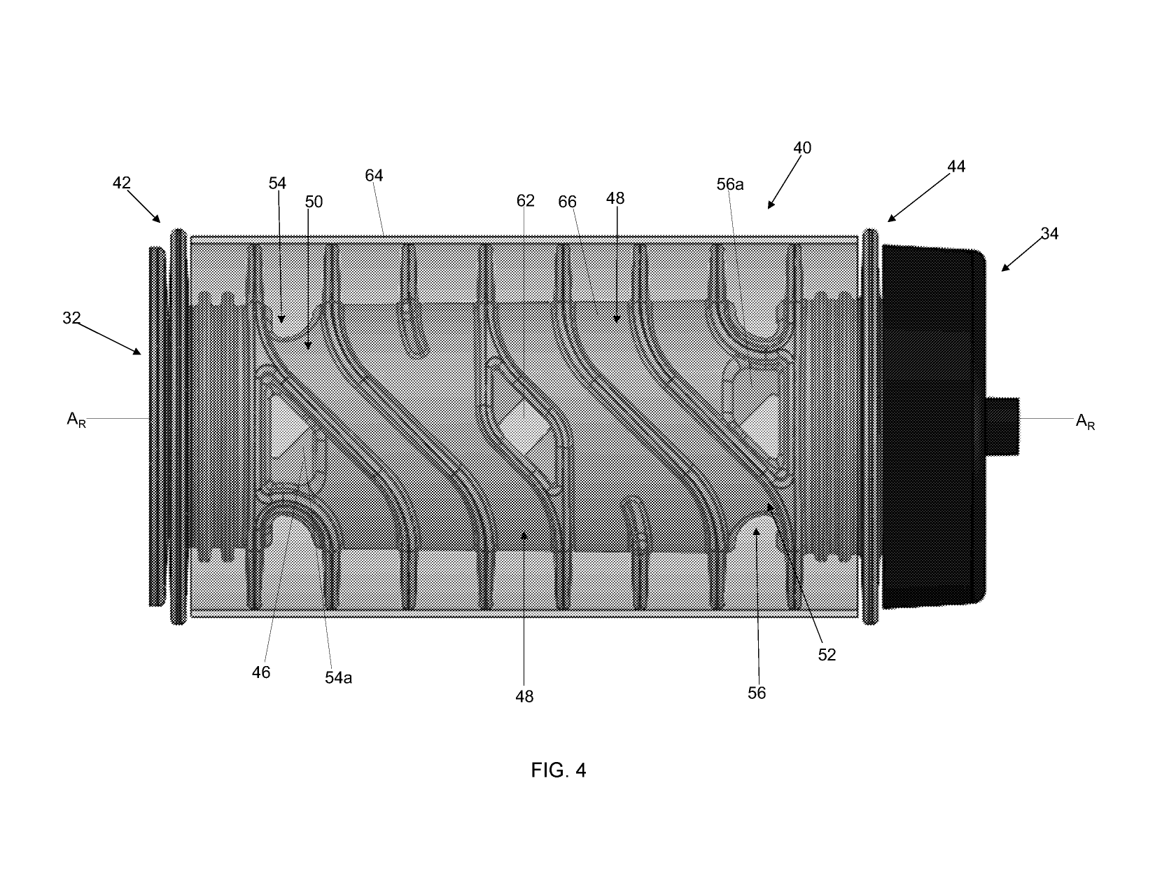

Referring to FIG. 4, the first aperture 54 and the second aperture 56 may be defined by the body 40 within or adjacent to a path 66 of the helical conduit 48. The first aperture 54 may be defined at or adjacent to the first end 50 of the helical conduit 48 and the second aperture 56 may be defined at or adjacent to the second end 52 of the helical conduit 48. The body 40 may define additional apertures 54a, 56a corresponding to additional helical conduits 48 in a configuration in which the body 40 includes a plurality of helical conduits 48. For example, in a configuration in which the body 40 includes a second helical conduit 48, the body 40 defines a third aperture 54a and a fourth aperture 56a corresponding to the second helical conduit 48, in a manner that is the same or substantially similar to the first helical conduit 48 and the corresponding first aperture 54 and the second aperture 56, respectively. In examples, the body 40 may also define a window 62 to allow a user to view the cultured tissue within the reservoir 46. The window 62 may be positioned between the first end 42 and the second end 44 of the body 40. The body 40 may also include a sheath 64 bound to the helical conduit 48 and, thus, the body 40, which is configured for gas exchange. To this end, the sheath 64 may be constructed from a gas-permeable material, such as polydimethylsiloxane (PDMS). It is contemplated that the sheath 64 may be bound to the body 40 by any process compatible with the perfusion vessel 10 (e.g. stretching, heat-shrinking, thermal welding, clamping, gluing, and/or the like).

The helical conduit 48 is configured to receive the culture medium and to facilitate movement of the culture medium between the first end 42 of the body 40 and the second end 44 of the body 40 during rotation of the body 40. In particular, the helical conduit 48 is configured to facilitate movement of the culture medium between the first aperture 54 and the second aperture 56 during rotation of the body 40. To this end, the helical conduit 48 is configured to function utilizing a principal of displacement, in a manner similar to an Archimedes screw. To this end, during rotation of the body 40 in the first direction, the second end 52 of the helical conduit 48 is configured to scoop up culture medium surrounding the body 40. Additionally or alternatively, during rotation of the body 40 in the first direction, the second end 52 of the helical conduit 48 is configured to scoop up culture medium that has exited the reservoir 46 through the second aperture 56. As the body 40 continues to rotate, culture medium scooped up by the second end of the body 40 is carried along the path 66 of the helical conduit 48 and, thus, the body 40, from the second end 52 of the helical conduit 48 toward the first end 50 of the helical conduit 48. Culture medium reaching the first end 50 of the helical conduit 48 is discharged from the helical conduit 48 through the first aperture 54. The culture medium passes through the first aperture 54 for entry into the reservoir 46 of the body 40. Additionally or alternatively, the culture medium passing through the first aperture 54 enters the flow port 58 for distribution into the reservoir 46. The culture medium entering the reservoir 46 is perfused to the cultured tissue, before again exiting the reservoir 46 through the second aperture 56, to again be scooped up by the second end 52 of the helical conduit 48. In this manner, the culture medium is automatically recirculated through the perfusion vessel 10 and, thus, to the cultured tissue. It is contemplated that the movement of culture medium through the perfusion vessel 10 is reversed, moving from the first end 50 of the helical conduit 48 toward the second end 52 of the helical conduit 48, during rotation of the body 40 in the second direction and/or positioning of the flow port 58 at the second end 44 of the body 40.

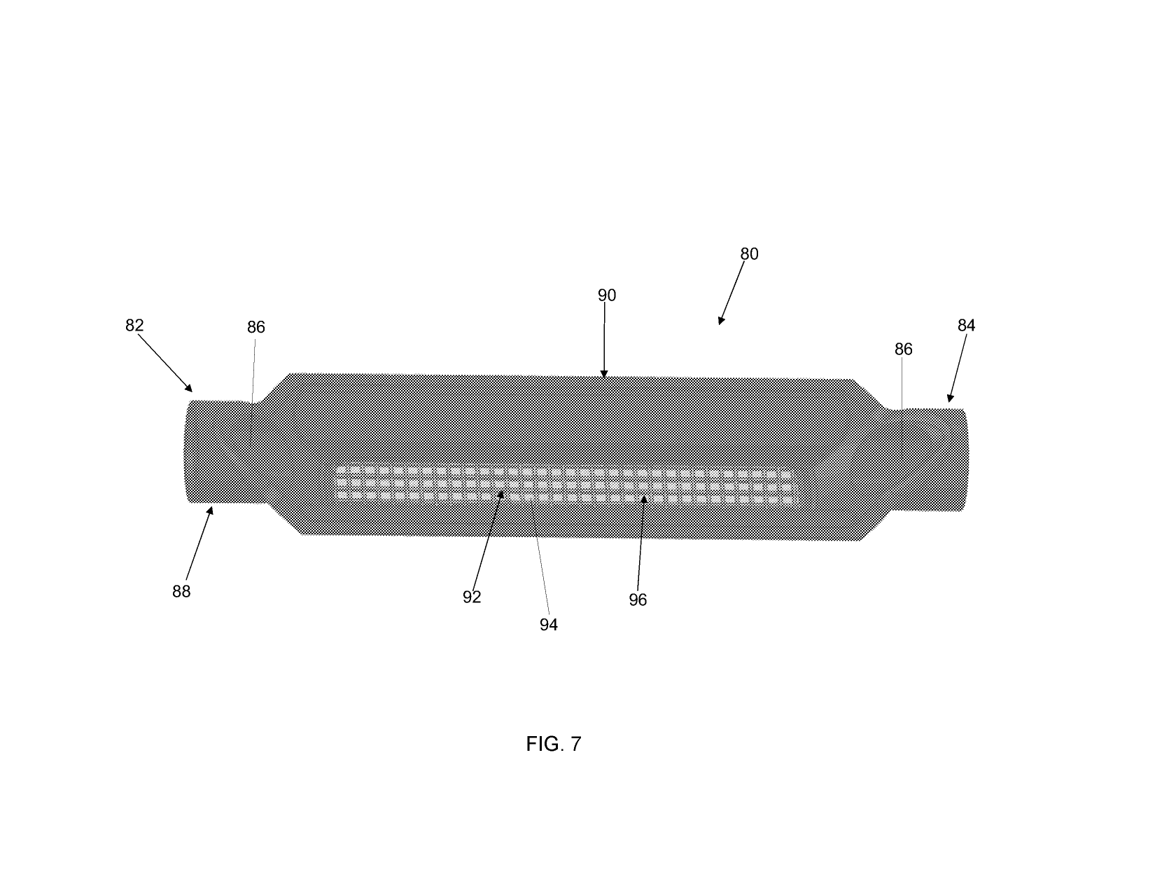

As shown in FIGS. 7-11, the support 80 extends axially between a first end 82 and a second end 84 and is configured to hold the cultured tissue. The support 80 is configured to be received within the reservoir 46 of the body 40, so as to be perfused with culture medium entering the reservoir 46 through the first aperture 54 and/or the flow port 58. It is contemplated that the second engagement member 88 of the support 80 may be included at or adjacent to one or more of the first end 82 and the second end 84 of the support 80, such that the support 80 is engaged by the body 40 at the first end 82 and second end 84 of the support 80, thereby suspending the support 80 within the reservoir 46 of the body 40. The support 80 may also include one or more flow ports 86, at or adjacent to one or more of the first end 82 of the support 80 and the second end 84 of the support 80, configured to be in communication with one or more of the flow ports 58 of the body 40 and/or the endcap 32, the first aperture 54 of the body 40, and the second aperture 56 of the body 40, respectively. In this manner, the support 80 may be configured to direct flow of the culture medium perfused through one or more of the flow ports 58 of the body 40 and/or the endcap 32 and the first aperture 54 of the body 40 through the reservoir 46 of the body 40. Additionally, in this manner, the support 80 may be configured to direct flow of the culture medium perfused through the reservoir 46 of the body 40 to the second aperture 56 of the body 40 and, thus, to the helical conduit 48 for recirculation of the culture medium. However, due to rotation of the body 40, it is contemplated that the culture medium may be perfused into the reservoir 46 of the body 40 directly through one or more of the flow ports 58 of the body 40 and/or the endcap 32 and the first aperture 54 of the body 40, through the reservoir 46 of the body 40, and out of the reservoir 46 of the body 40 directly through the second aperture 56 of the body 40.

In exemplary embodiments, the support 80 may float on its own, obviating the need for connection between the support 80 and body 40. In this case, the support 80 may be made of a suitably buoyant polymer, for example a polypropylene.

The support 80 includes a base 90 extending between the first end 82 and the second end 84 of the support 80. The support 80 includes a platform 92 extending across the base 90. The platform 92 is configured to hold and/or contain the cultured tissue. To this end, the platform 92 includes one or more well 94 configured to hold cellular aggregates, spheroids, organoids, small tissues, and/or the like. The well 94 may have a width within a range of 50 μm to 3 mm and a height within a range of 50 μm to 3 mm. In examples, the platform 92 may include a plurality of wells 94; however, the support 80 will be described with reference to “the well 94,” unless reference to a plurality of wells 94 is otherwise necessary. The support 80 includes a permeable membrane 96 extending across the platform 92. It is contemplated that the permeable membrane 96 is configured for cells and/or the cultured tissue to adhere to, thereby forming a confluent layer across the permeable membrane 96, within the well 94. The permeable membrane 96 may be configured to establish an interstitial flow resistance and/or interstitial flow rate of the culture medium perfused through the permeable membrane 96. In examples, the interstitial flow rate established by the permeable membrane 96 is within a range of 0.05 µm/sec to 5 µm/sec. In examples, the permeable membrane 96 may extend across the platform 92, at a position beneath the platform 92. The permeable membrane 96 may be constructed from a durable, porous material, such as track-etched polycarbonate or polyethylene terephthalate (PET). The permeable membrane 96 may have a pore size within a range of 1 µm to 30 µm. The permeable membrane 96 may be bound to the platform 92 by any process compatible with the perfusion vessel 10 (e.g. stretching, heat-shrinking, thermal welding, clamping, gluing, and/or the like).

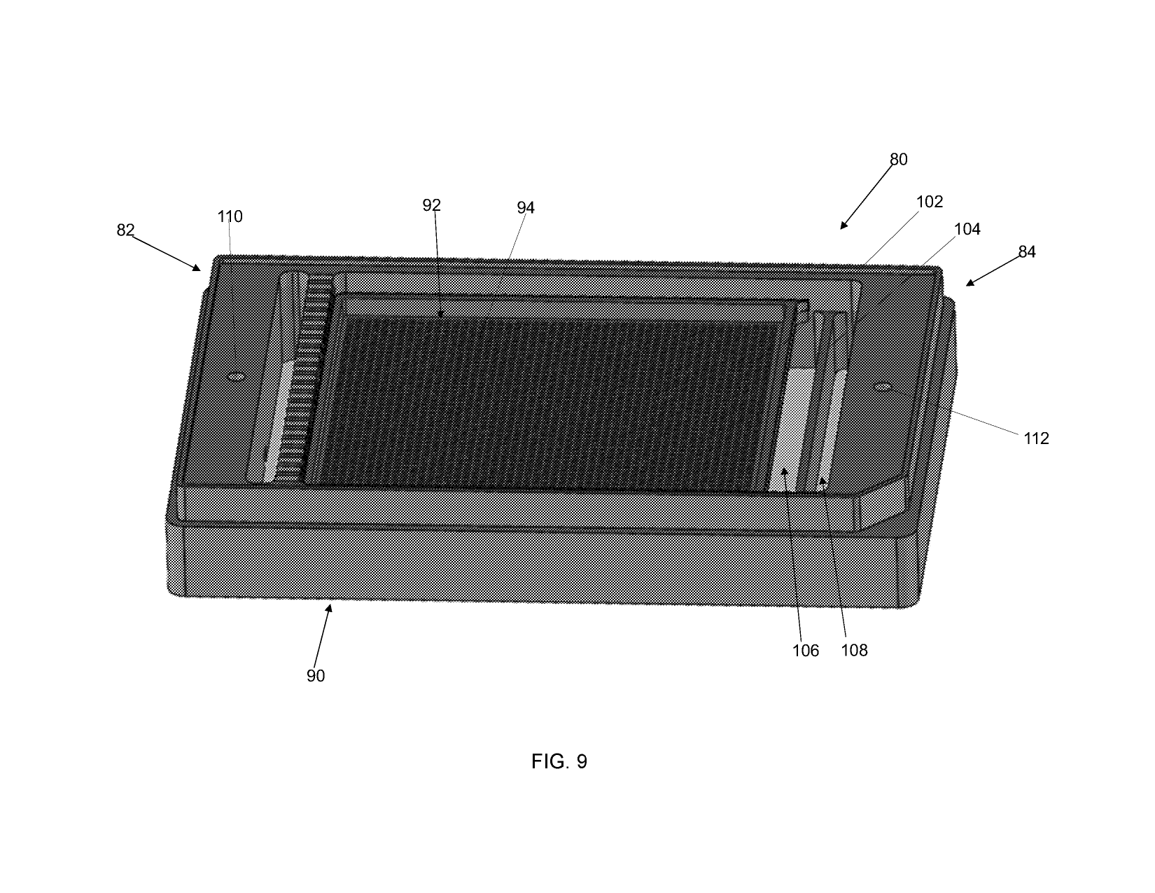

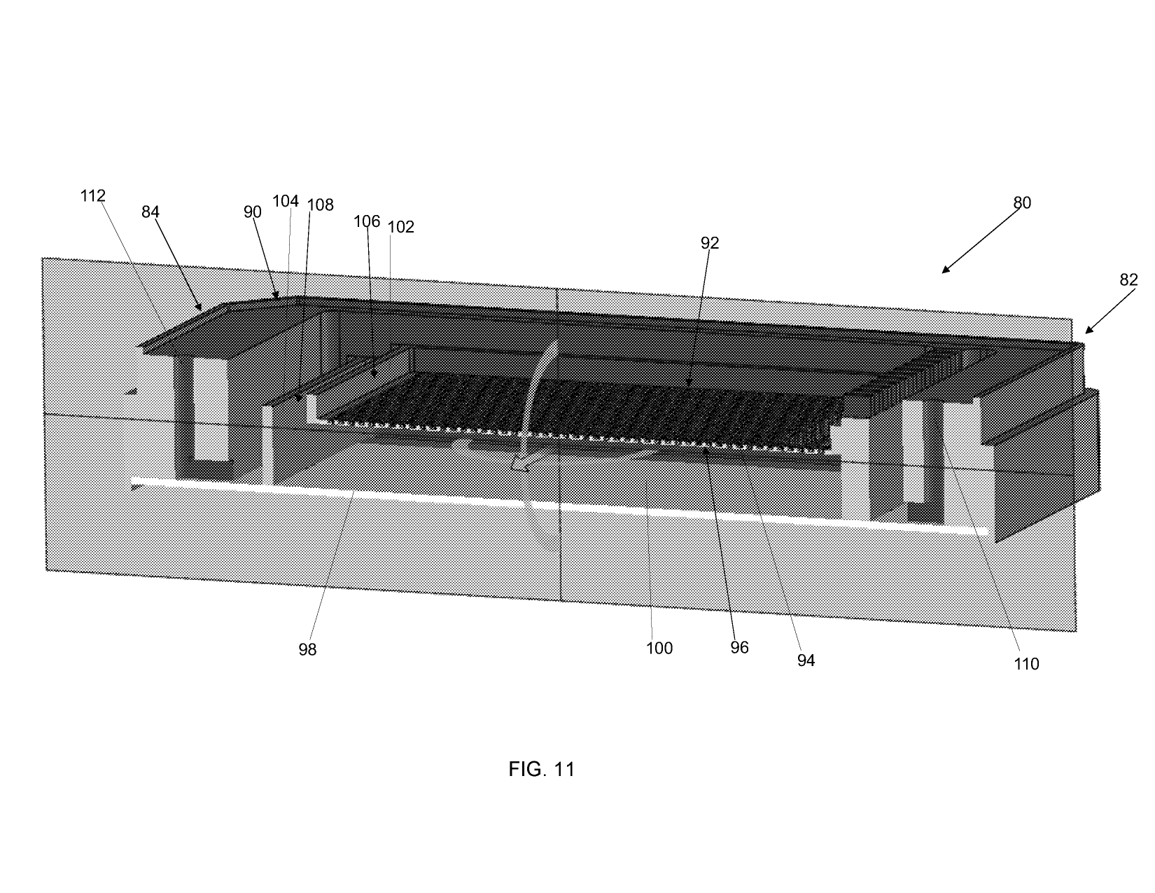

As shown in FIGS. 9-11, additionally or alternatively, the support 80 may be configured to establish a pressure head-limited perfusion of the culture medium through the reservoir 46 of the body 40. Referring to FIG. 11, the support 80 may include a floor 98 extending across a lowermost portion of the base 90. The platform 92 may be elevated and/or distanced from the floor 98 of the base 90, such that a cavity 100 is defined between the platform 92 and the floor 98. The platform 92 may also be an independent component that can be removed from the base 90. The cavity 100 may be configured for collection of the culture medium perfused into the reservoir 46 of the body 40. The support 80 may include a first wall 102 and a second wall 104 extending from the support 80, perpendicular or substantially perpendicular to the platform 92 and the permeable membrane 96. In such a configuration, the first wall 102 extends from a position elevated and/or distanced from the floor 98 of the base 90, at or adjacent to the platform 92. The second wall 104 extends directly from the floor 98 of the base 90. The first wall 102 and the second wall 104 extend from a position that is outward of the platform 92. It is contemplated that the terms “outward” and “inward” as used herein may be understood with respect to a center of the perfusion vessel 10. In this manner, one or more of the first wall 102 and the second wall 104 further define the cavity 100. In examples, the first wall 102 extends from a position that is inward of the second wall 104, between the platform 92 and the second wall 104. The first wall 102 extends to a first height, the second wall 104 extends to a second height, and the first height of the first wall 102 is greater than the second height of the second wall 104. The platform 92 extends across the support 80 at third height that is less than the first height of the first wall 102. In this manner, the culture medium may be collected in the cavity 100, forming a first column 106 of the culture medium contained by the first wall 102 at or adjacent to a position above the second wall 104 and the platform 92, due to the first wall 102 extending to the first height that is greater than the second height of the second wall 104 and the third height of the platform 92. Additionally, the culture medium may be collected in the cavity 100, forming a second column 108 of the culture medium contained by the second wall 104 at or adjacent to a position beneath the platform 92 and/or the first wall 102, due to the first wall 102 being elevated and/or distanced from the floor 98. Any overflow of the culture medium contained by the first wall 102 in the first column 106 may spill over the first wall 102 into the culture medium contained by the second wall 104 in the second column 108.

In this manner, due to a difference in the first height of the first wall 102 and the second height of the second wall 104 (i.e. a pressure head), as well as overflow of the culture medium from the first column 106 of culture medium to the second column 108 of the culture medium, the height of the first column 106 of the culture medium corresponds to an actual pressure exerted by the first column 106 of the culture medium on the floor 98 of the base 90, thereby enabling the support 80 to be configured to establish a pressure head-limited perfusion of the culture medium through the reservoir 46 of the body 40. In examples, the difference in height between the first height of the first wall 102 and the second height of the second wall 104 and/or the height of the first column 106 of culture medium and the height of the second culture medium (i.e. the pressure head) may be within a range of 0.25 mm to 25 mm.

By understanding and/or harnessing the pressure of the culture medium within the reservoir 46 of the body 40, a behavior of the culture medium within the perfusion vessel 10, direction and rate of flow of the culture medium within the perfusion vessel 10, an energy content of the culture medium within the perfusion vessel 10, and/or the like can be determined and/or regulated, thereby increasing control over fluid mechanics within the perfusion vessel 10. Additionally or alternatively, by understanding and/or harnessing the pressure of the culture medium within the reservoir 46 of the body 40, in combination with establishing an interstitial flow resistance and/or interstitial flow rate of the culture medium perfused through the permeable membrane 96, control over fluid mechanics within the perfusion vessel 10 may be further increased. For example, a constant pressure gradient may be established across the permeable membrane 96. Additionally or alternatively, for example, a perfusion flow rate of the culture medium through the reservoir 46 of the body 40 may set to be lower than a recirculation flow rate of the culture medium from the first aperture 54 toward the second aperture 56 along the helical conduit 48.

It is contemplated that the culture medium may be perfused through the reservoir 46 and into the cavity 100 through the flow port 58 of the body 40 and/or the endcap 32. Additionally or alternatively, it is contemplated that the support 80 may incorporate a pump (not shown) configured to input and/or output the culture medium to and/or from the cavity 100. In such a configuration, the base 90 of the support 80 may further define an inlet 110 and/or an outlet 112 in communication with the pump and the cavity 100.

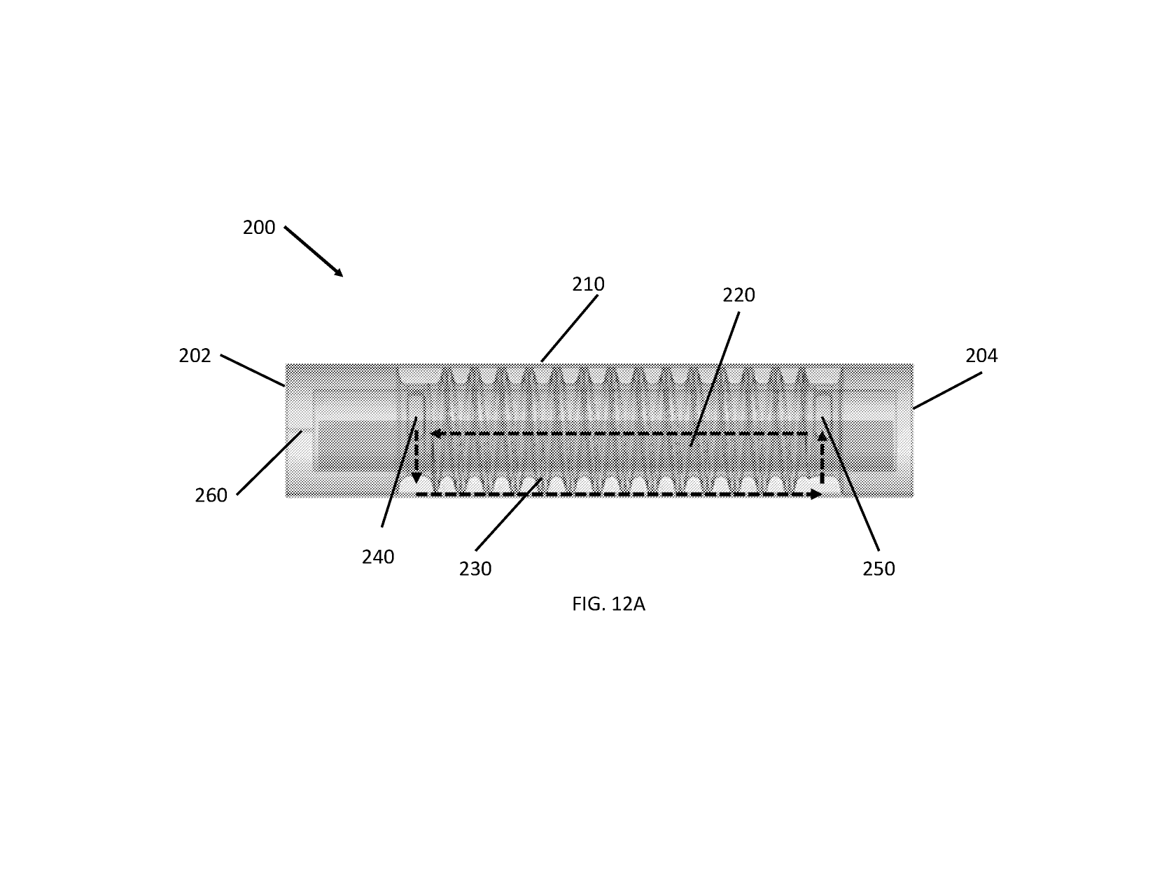

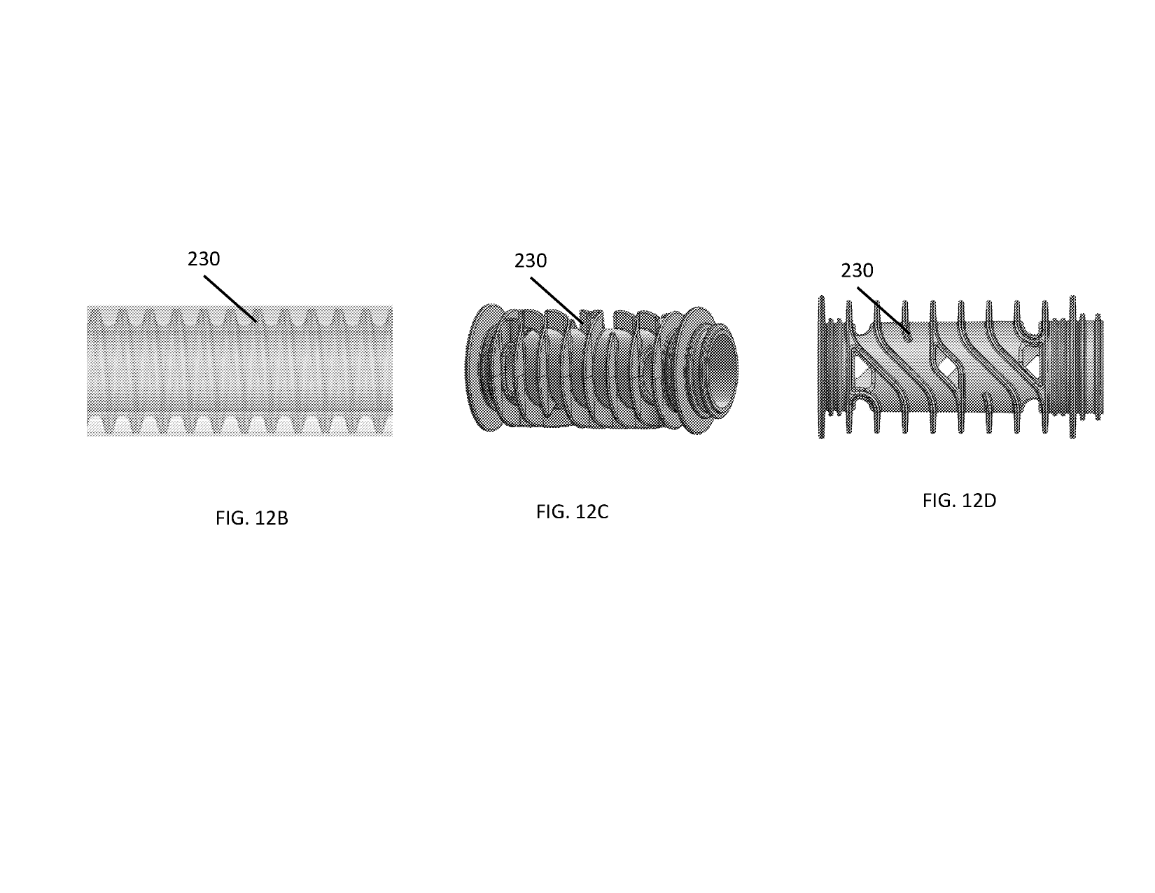

FIG. 12A shows a rotation driven media pump, generally designated by reference number 200, according to another exemplary embodiment of the present invention. The media pump 200 is generally configured to displace fluid along the longitudinal axis of the pump 200 so that the fluid can be recirculated. The media pump 200 has a first end portion 202 and a second end portion 204 and includes an outer sheath 210, an internal reservoir 220, a helical channel 230 disposed around the internal reservoir 220, a first port 240 disposed at one end portion of the helical channel 230 and in communication with the internal reservoir 220 and a second port 250 disposed at a second end portion of the helical channel 230 and also in communication with the internal reservoir 220. A transfer port 260 may be disposed at one or both ends of the media pump 200 for injection or retrieval of the media.

The media pump 200 may be caused to rotate, resulting in circulation of the media between the end portions of the pump 200. In this regard, the helical channel 230 is configured to facilitate movement of the media between the first port 240 and the second port 250 during rotation of the media pump 200. To this end, the helical channel 230 is configured to function utilizing a principal of displacement, in a manner similar to an Archimedes screw. As indicated by the arrows in FIG. 12A, as the helical channel 230 rotates, media at the second end portion 204 of the media pump 200 is scooped up into the second port 250 and made to travel within the internal reservoir 220 towards the first end portion 202 of the media pump 200. At the first end portion 202, the media is released through the first port 240 so as to be once again external to the internal reservoir 220, where the media travels back towards the second end portion 204. Reversal of rotation of the media pump 200 will cause the media to flow in the opposite direction. It was observed that when the device is completely filled with media, flow cannot develop. Without being bound by theory, it is believed that two generally immiscible fluids of differing densities, for example air and water, are required to facilitate flow through the channel during rotation of the device.

As shown in FIGS. 12B-12D, the helical channel 230 may be formed in various ways. For example, as shown in FIG. 12B, the helical channel 230 may be a simple spiral, with the sheath formed in spiral form or with tubing wrapped in a spiral form, as shown in FIG. 12C, the helical channel 230 may be made up of an incremental spiral (for injection molding), or as shown in FIG. 12D, the helical channel 230 may be made up of multiple joined and/or split paths (to enhance mixing and/or provide a port or window directly into the internal reservoir 220).

In exemplary embodiments, the helical channel 230 may be joined to the outer sheath 210. In this regard, the sheath 210 can be elastomeric and stretched over the helical channel, thereby sealing the helical channel in compression, the sheath can be made of heat shrinkable material, the sheath can be a tube welded to the screw geometry via known methods (e.g., heat, ultrasonic, laser, friction welding), the sheath can compress the screw component, which may be made of a compliant thermoplastic material or elastomer, the sheath can have a loose fit over the screw component (in this regard, the device may still function even with small gaps between sheath and screw) and/or adhesives may be used to bond the two components together.

In exemplary embodiments, the helical channel 230 may be fabricated using various methods, including, for example, injection molding, machining, blow molding/thermoforming, additive manufacturing (e.g.,3D printing), or casting/reaction molding, to name a few.

The inventive rotary perfusion approach provides a number of advantages, including: no “moving parts” or tubing/pumps; can be made as a singular body as low cost consumable; low shear (with low rpm); no dead volume (all medium is recirculated); highly parallelizable (on a multi-level roller or rotating holder); closed system (with appropriate ports for aseptic connection); can utilize high area gas permeable boundaries for efficient equilibrium with external environment (e.g., controlled by a cell culture incubator).

In exemplary embodiments, the rotational speed of the inventive perfusion vessel and media pump should be limited to avoid instability. In general, without being bound by theory, centrifugal forces on liquid at high rotations per minute (RPM) will conflict with the forces of gravity to maintain consistent perfusion rates. For example, a device of diameter ~3cm with spiral channel diameter of ~4mm will become unpredictable at speeds greater than ~150-200RPM. This transition state is highly dependent on surface tension properties of the material, medium, temperature, and fill volume. There is no lower bound for RPM (approaching 0)

In exemplary embodiments, the internal channel diameter should be large enough to optimize flow and rotation speeds. In this regard, without being bound by theory, larger internal channel sizes will behave more consistently as the capillary effect is minimized. In this situation, there is a lower bound for the channel size, which is also dependent upon surface tension properties of the material and medium, as well as channel geometry, fill volume, and rotation speed. For a device made from polypropylene of overall diameter ~3cm, for example, with a contact angle of ~100 degrees and medium with a high surface tension of ~70 dyne/cm at 37C temperature, a channel diameter of at least 1mm is necessary to enable flow during low-speed rotation, with larger diameter channels being preferred to allow for a wider range of rotation speeds. These channel specifications would adjust depending on the material used, medium used, operating temperature, overall device size, and apparent gravity.

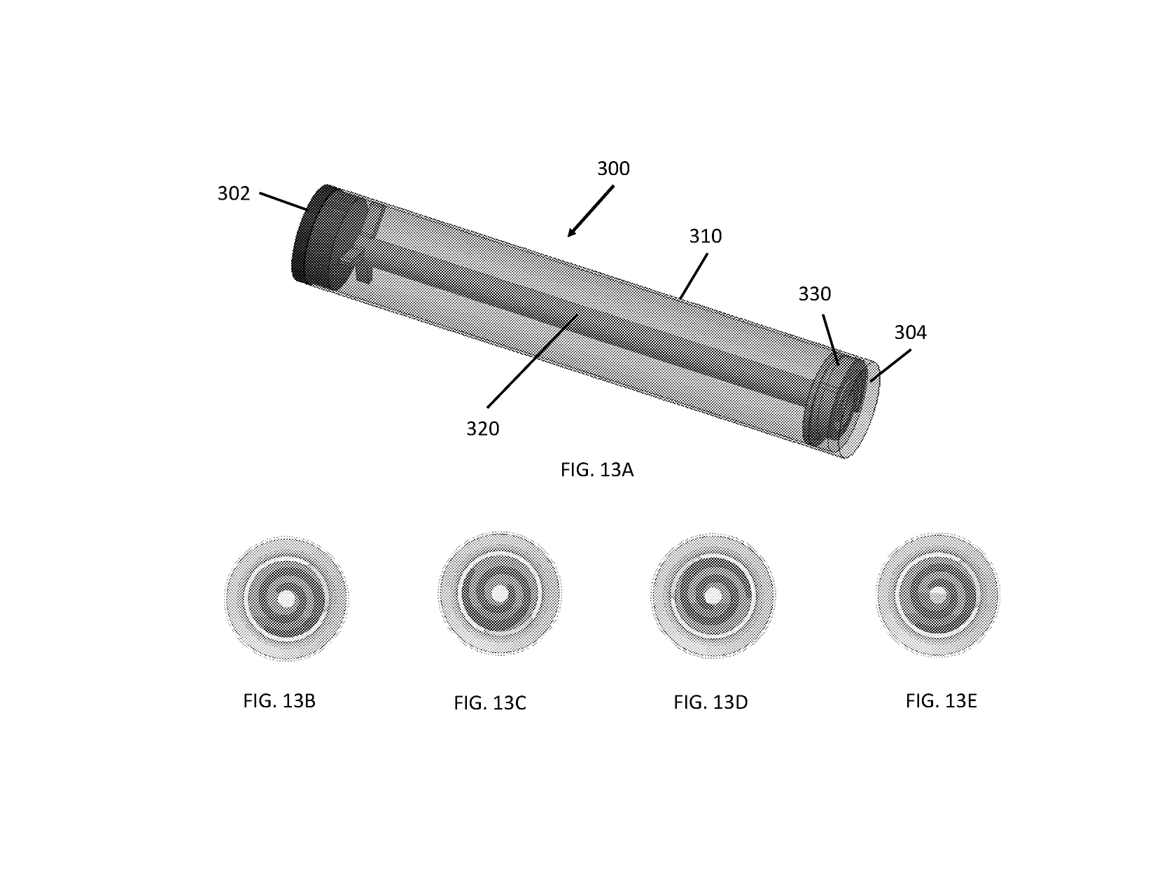

FIG. 13A shows a rotation driven media pump, generally designated by reference number 300, according to another exemplary embodiment of the present invention. The media pump 300 is generally configured to displace fluid along the longitudinal axis of the pump 300 so that the fluid can be recirculated. The media pump 300 has a first end portion 302 and a second end portion 304 and includes an outer sheath 310, an internal tube 320, a helical structure 330 disposed at one end of the tube 320 and in communication with the internal tube 320, and one or more ports (not shown) along the length of the tube 320. With rotation of the media pump 300, the helical structure 330 (acting as a “scoop pump”) scoops up media from the outer diameter of the media pump 300 and into the tube 320, which is located along the central axis of the pump 300. The tube 320 provides a path for the media to flow along the central axis of the pump 300 and through the ports along the tube 320. More specifically, as shown in FIGS. 13B-13E, media enters the spiral structure 330 (FIG. 13B) and follows the spiral channel of the spiral structure 330, continues to follow the spiral channel as the tube 320 rotates (FIGS. 13C, 13D) so as to enter the tube 320, and then finally exits the tube 320 through the one or more ports as more media enters the spiral channel (FIG. 13E). In this manner, media can be continuously circulated through the media pump 300 as the pump 300 rotates. As described with reference to the embodiment shown in FIGS. 1-3, the inventive perfusion vessel may include a support 80 or other type of insert. However, such an insert is not necessary, and in other exemplary embodiments the cells/aggregates may recirculate in suspension or bulk tissues (natural or engineered, with or without vascularization) may be simply included within the vessel. In other exemplary embodiments, the perfusion vessel or any of the rotational media pumps described herein may include any type of insert as needed to accommodate physiologic environmental needs of cells and/or tissues. For example, the insert may include one or more of the following: microspheres in recirculation (with cells attached to their surface); a packed bed approach (e.g., larger spheres, either solid or porous); a thin-film/mesh rolled/spiral structure either flat or overlaid with porous niches for aggregates; micropatterned inserts for aligned cell/cell-sheet growth; an internal porous mesh-bound bag; a bulk porous scaffold (e.g., cylinder of sponge-like geometry); reticulated foam; trabecular bone; and 3D printed mesh structures, to name a few.

In exemplary embodiments, the insert (such as the support 80) may be a boat-like component that is configured to float in the liquid media held in the inventive device, preferably without rotation. In this regard, the insert may be configured with a porous membrane at its base, with one or more of the following features: wells of various geometries formed above the membrane to influence size of local environment for aggregates, spheroids, organoids, etc.; smaller wells/cavities to promote cellular self-assembly; layered transwell geometries for controlled cell-cell stimulation or increased surface area; a membrane at a top of the insert so as to contain cells in a pouch with a single inlet port; layering to obtain size exclusive regions (e.g., t-cells bounded by 5 um membrane with feeder cells outside this); and singular or multiple floating inserts, to name a few.

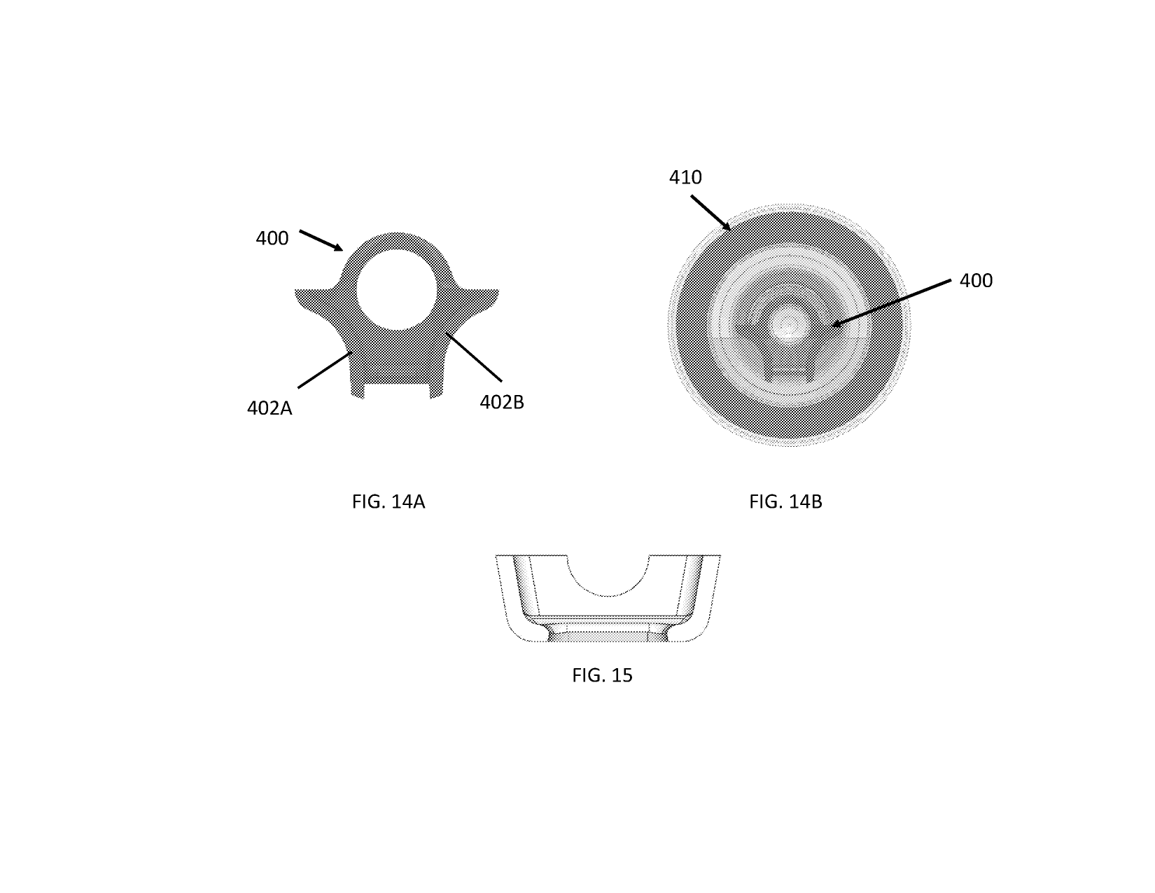

FIGS. 14A and 14B are cross-sectional views of an insert, generally designated by reference number 400, according to an exemplary embodiment of the present invention, with FIG. 14B showing the insert 400 disposed within a perfusion vessel 410. The insert 400 includes concave side surfaces 402A, 402B, resembling the hull of a ship or a center-board on a sailing vessel. Based on experimental observation, and without being bound by theory, it is believed that this specific geometry with more vertical features can more readily hold its upright orientation during device rotation, as opposed to, for example, a flat-bottomed or rounded bottomed geometry (an example of which is shown in FIG. 15) which does not have sufficient surface area orthogonal to the direction of rotation. In exemplary embodiments, the insert may be a multi-body construction in which a portion can be removed to facilitate downstream processes. For example, the size may be appropriate to fit within a standard well of various multi-well plates, or also a tissue processing cassette.

In exemplary embodiments, the insert may be provided with a surface coating, having one or more of the following characteristics or materials: hydrophobic; hydrophilic; molecules including proteins, peptides, antibodies, enzymes, and other target-specific molecules aimed at inducing an expected cellular response; coating of cells, “feeder-cells” (irradiated or non irradiated), and/or antigen presenting cells (naturally derived or engineered); biodegradable/time release coatings for slow addition of compounds of interest; and made up of hydrogels, to name a few.

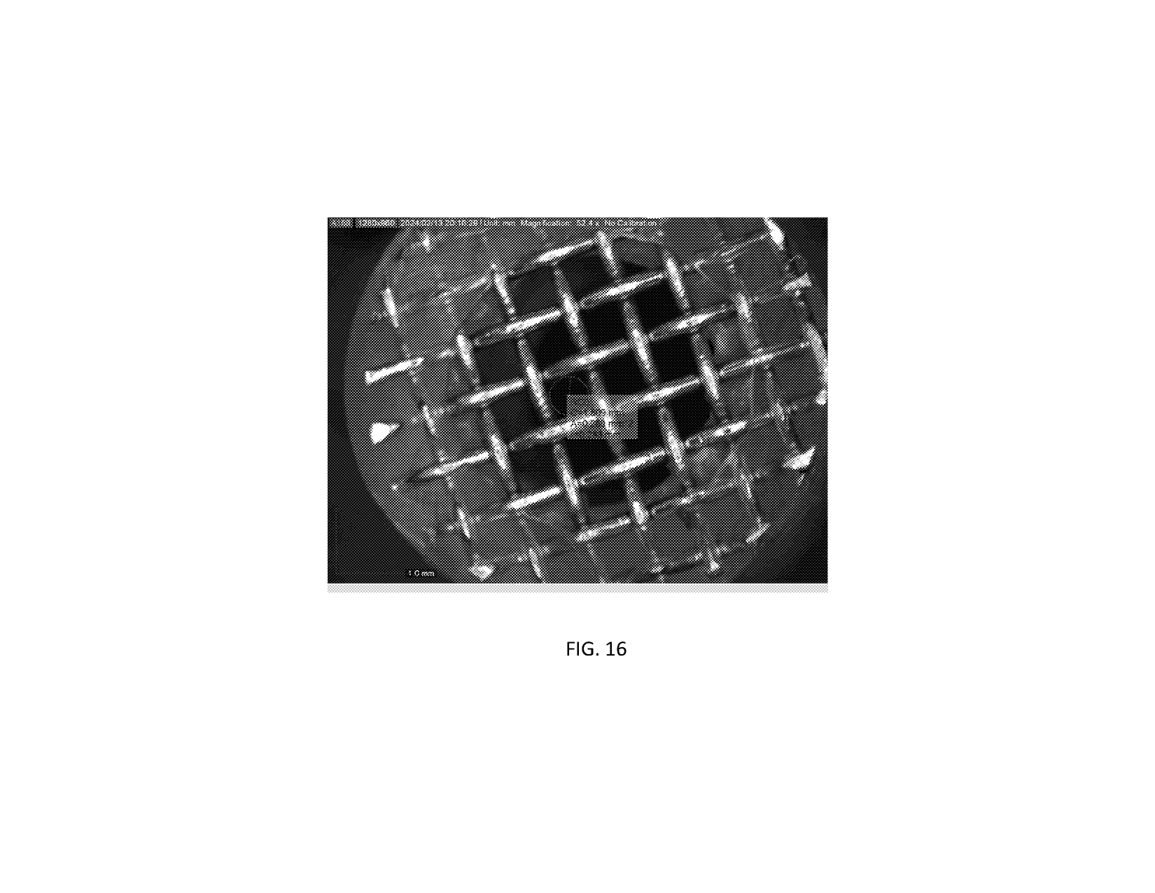

As shown in FIG. 16, in exemplary embodiments, any of the ports of the inventive device may include a mesh screen 500 configured to break up large aggregates. The mesh screen 500 may continuously maintain aggregates of a specified size range, use mechanical size-based passaging of cells for more continuous culture and/or be used to break up bulk tissue samples. The screen 500 may be overmolded on any of the ports or attached as a separate unit.

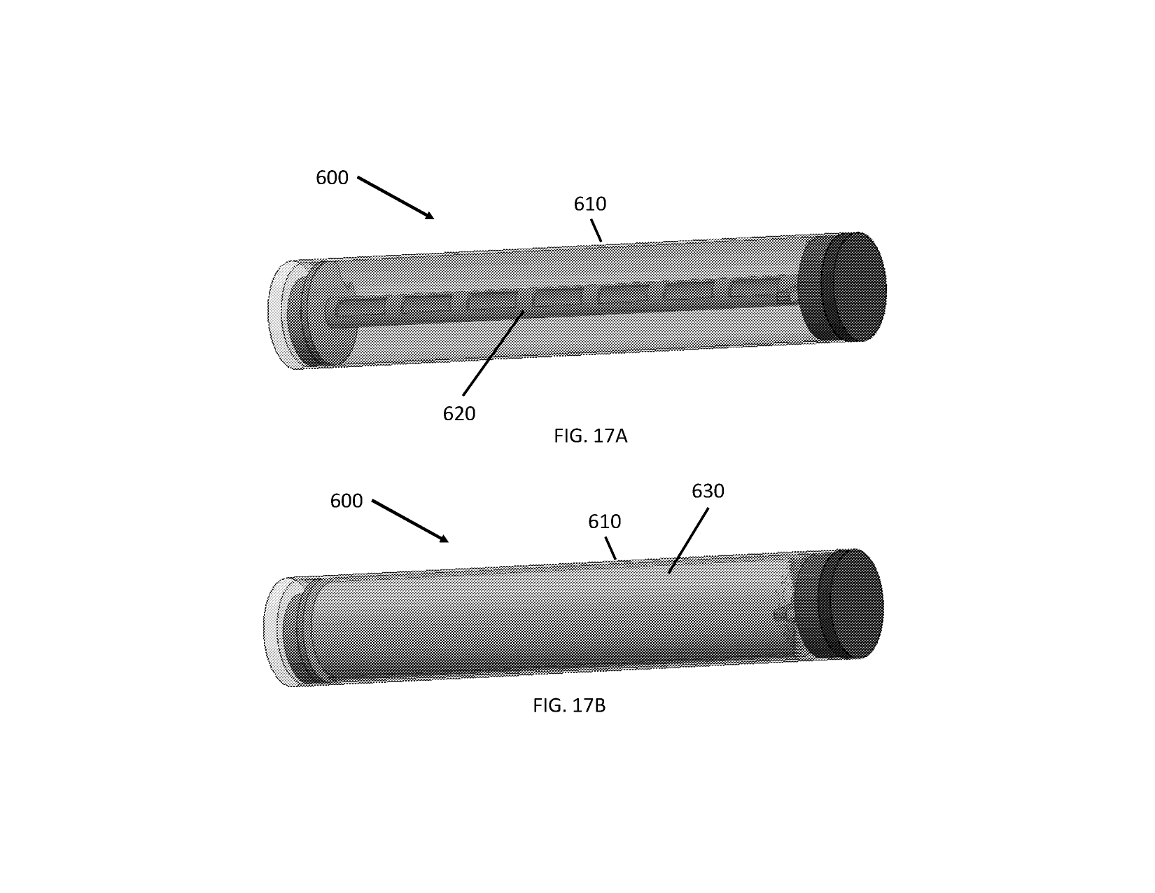

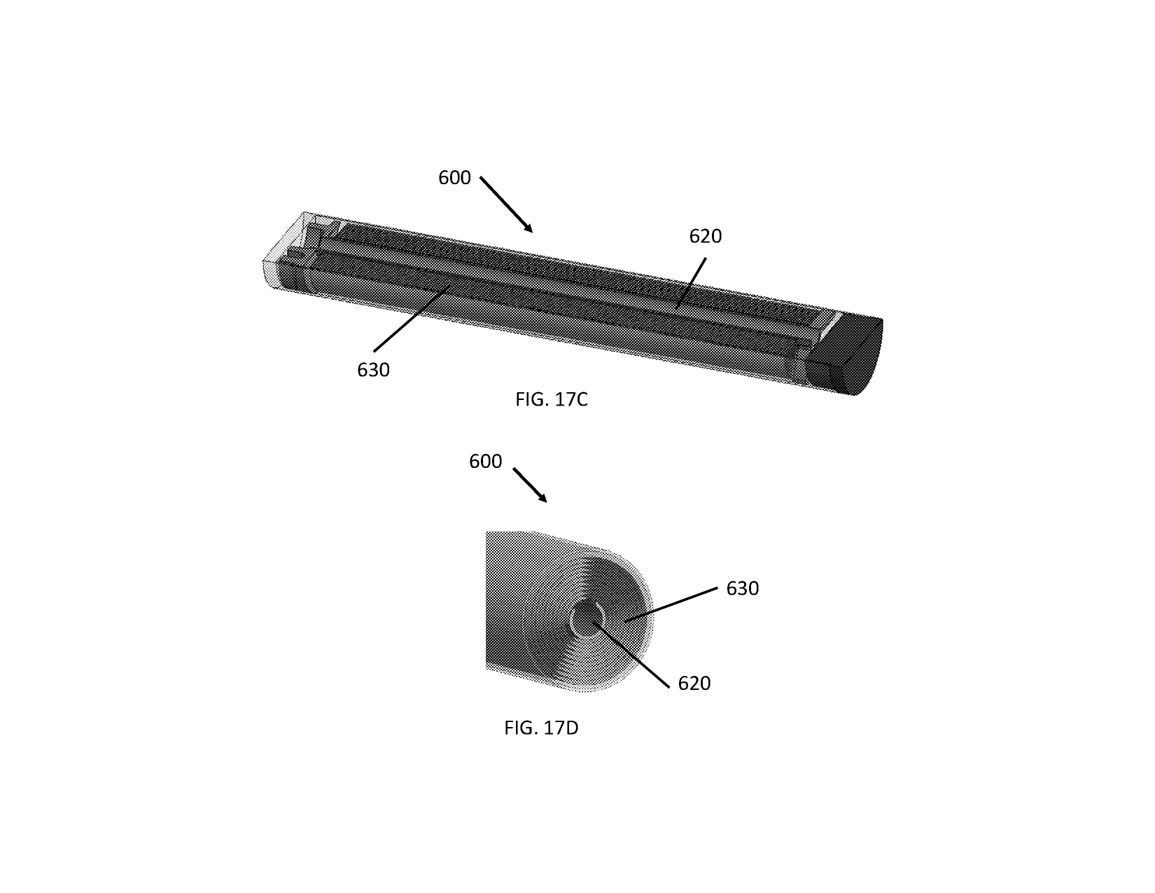

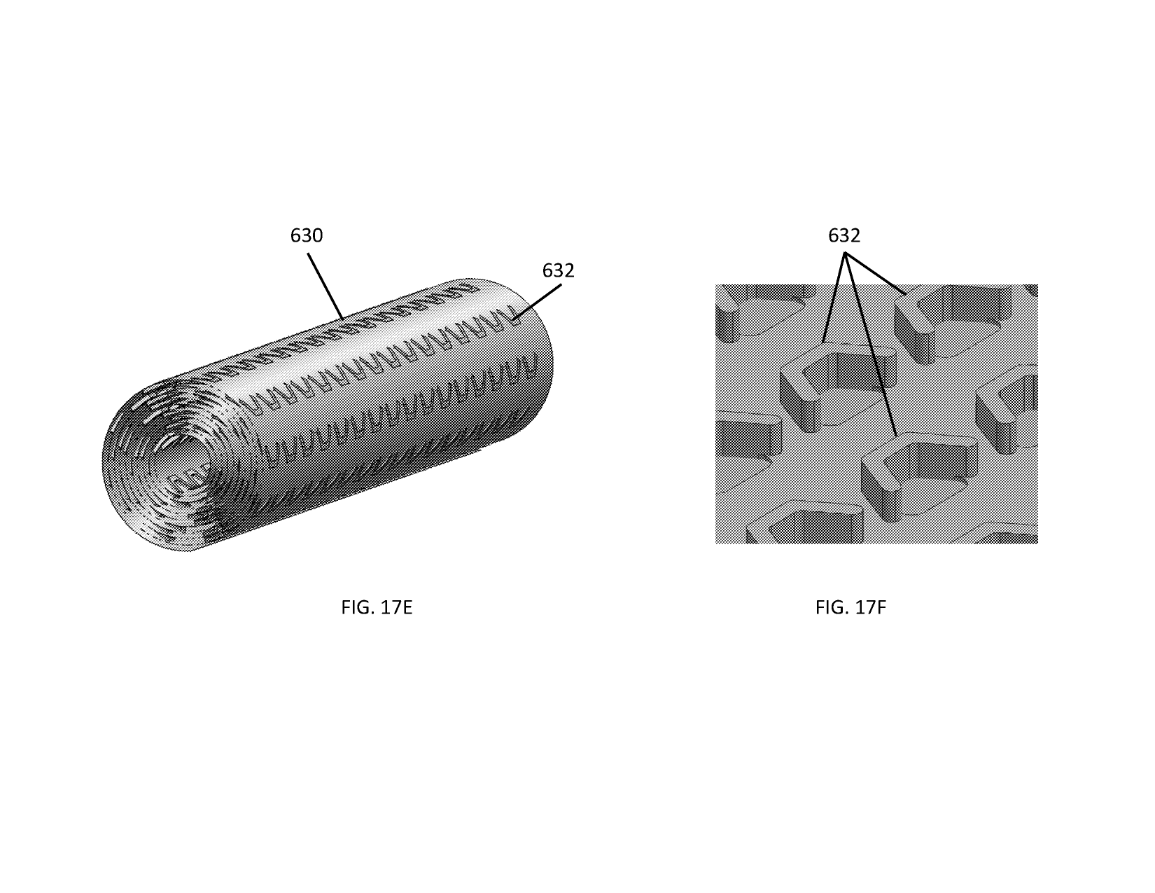

FIGS. 17A-17F show a rotational media pump, generally designated by reference number 600, according to another exemplary embodiment of the present invention. The media pump 600 includes an outer sheath 610, an internal reservoir 620 and a spiral wound film 630 disposed around the internal reservoir 620 (FIG. 17A shows the media pump without the spiral wound film 630 for ease of illustration). The internal reservoir 620 has one or more ports 622 disposed along its length which enable a fluid routing path between the internal reservoir 620 and the spiral wound film 630. The spiral wound film 630 has sufficient spacing between the spirals so that surface tension/capillary action forces do not impede free flow during rotation. In exemplary embodiments, the spiral wound film 630 itself can serve as a scoop-pump, or the media pump 600 may be provided with an additional scoop pump and/or external helical channels.

In exemplary embodiments, the spiral wound film 630 may incorporate features for application-specific use. For example, as shown in FIGS. 17E and 17F, the spiral wound film 630 may include traps 632 having a C-shape geometry to facilitate aggregation of cells in suspension up to a maximum size, defined by the walls of the traps 632.

In exemplary embodiments, any of the inventive perfusion vessels or media pumps described herein may include one or more application-specific interfacial features or modules derived from user workflow needs. The interfacial features may be fluid connections having one or more of the following characteristics: single per side; multiple per side; open; closed; fixed; freely rotating; filtered to prevent removal of cells; luer; barbed; compression; aseptic; and ‘closed’ luers (e.g., swabbable, closed until engaged with other gender), to name a few. The interfacial features may be configured to attach to various sized vials, such as, for example, cryovials and septum covered vials.

In exemplary embodiments, the inventive perfusion vessels or media pumps described herein may be rotated using rollers. Such rollers may be off-the-shelf rollers or have custom footprints. Roller mechanisms can be used in combination with a standard cell culture incubator or be setup as a stand-alone unit, incorporating one or multiple pull-out drawers within a unit that has built-in incubation capability (e.g., temperature, humidity, gas mixing). The standalone device may also include imaging via camera/objective assembly on an XY gantry above and/or below each set of rollers. The vessels may alternately be rotated by what is commonly referred to as a tube rotator, in the form of a vertically oriented carousel, which spins a rotor onto which multiple tubes can be fixed. Their preferred orientation in this case would be parallel to the axis of rotation which is also parallel to gravity.

In exemplary embodiments, the inventive perfusion vessels or media pump described herein may have applications related to cell culture or unrelated to cell culture. Examples of cell culture applications include: expansion and culture (2D, 3D, or microcarriers) of cell types, including mammalian (human, mouse, etc.), non-mammalian (insect, reptile, etc.), and other types of cells (plant, bacteria, yeast, fungus, etc.); different culture types, including single or multi-cell suspension, aggregate suspension, organoids, adherent, and 3D tissue; cell-produced factors (e.g., biologics, proteins, antibodies, exosomes, cytokines, mitochondria); therapeutic; disease modeling; screening; bio-banking; diagnostics; vaccine production; precision fermentation; microbial bioproduction; stem cell maintenance and differentiation; transduction; and local preparation of pre, pro, post-biotics for consumption or therapy (e.g., gut, skin, stool microbiome) to remove the need for addition of preservatives, to name a few. Examples of non-cell culture applications include: “accelerated ageing” of spirits (e.g., probiotic alcohol drinks); batch or continuous culture of bacteria and yeast based drinks; mixing; temperature control (heating/cooling) of fluids (high conductive transfer area); and removal of contaminants from solutions (e.g., wastewater treatment), to name a few.

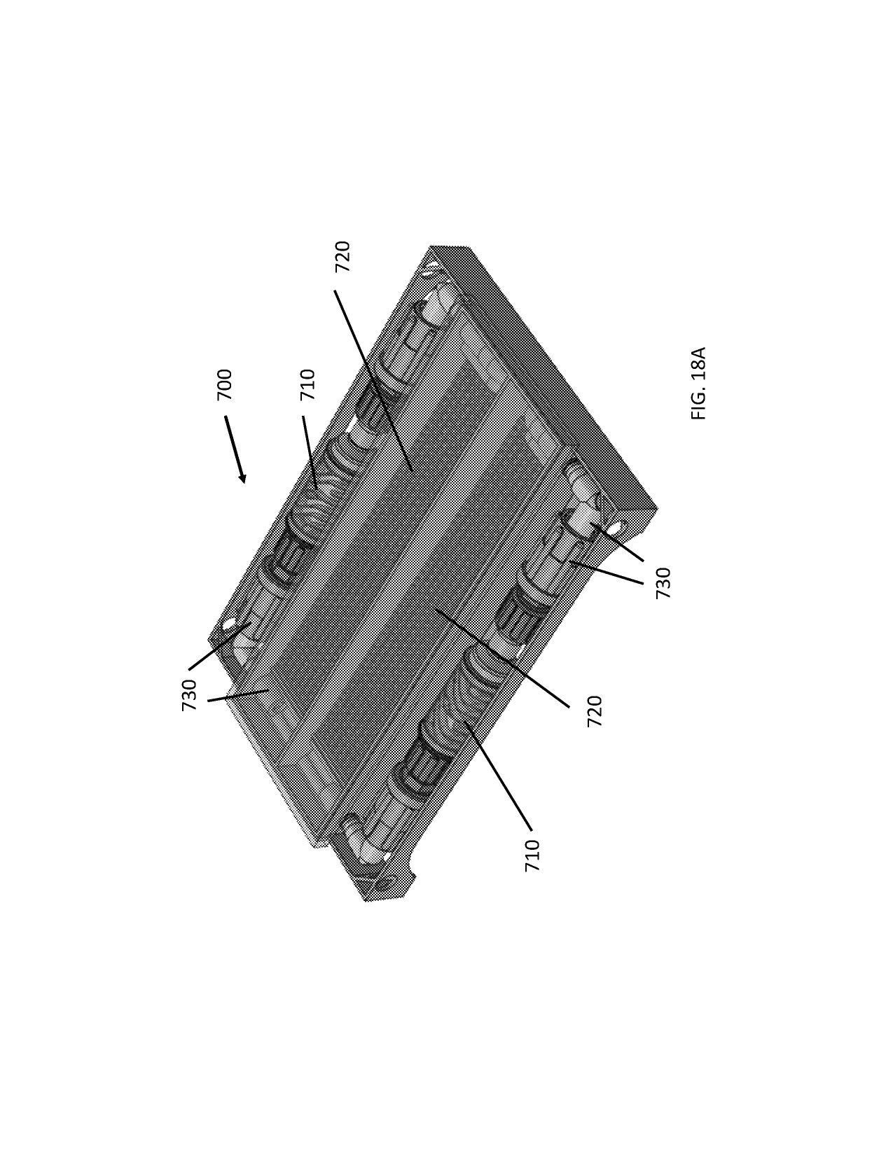

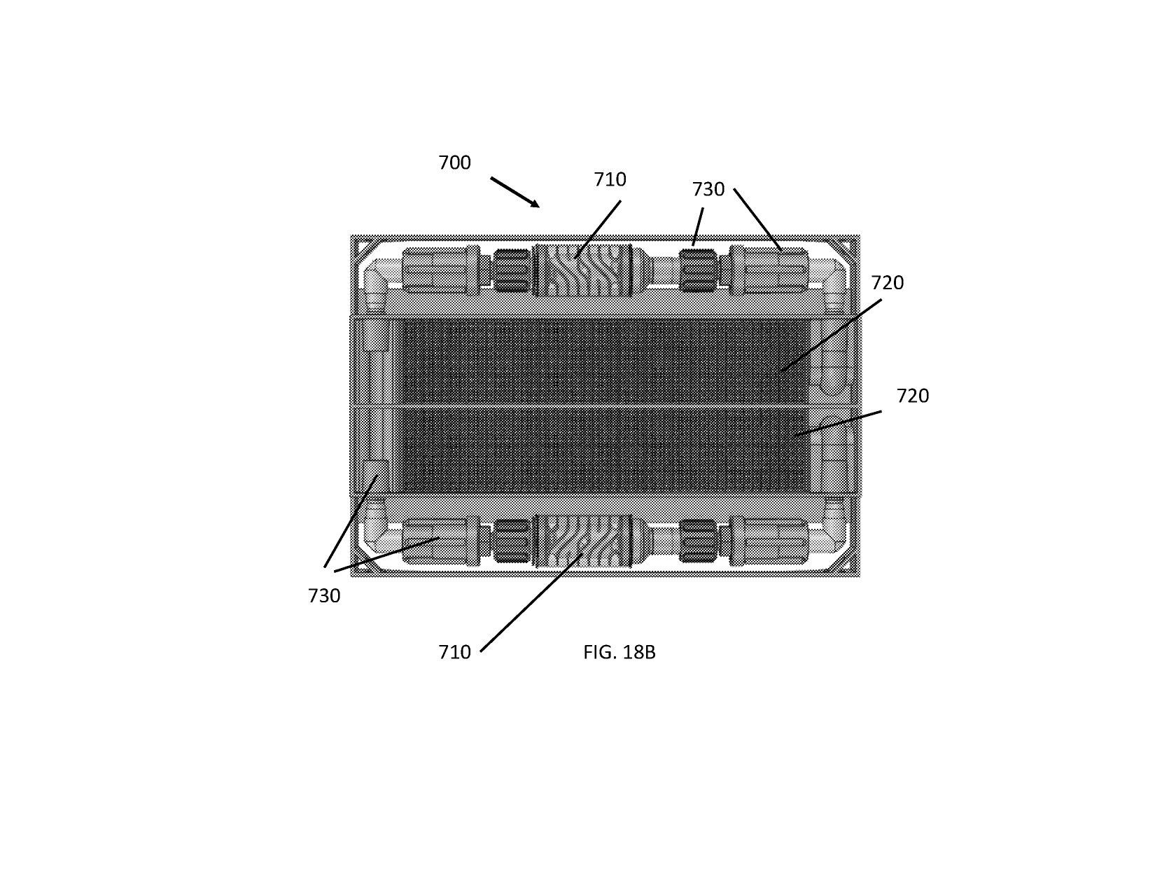

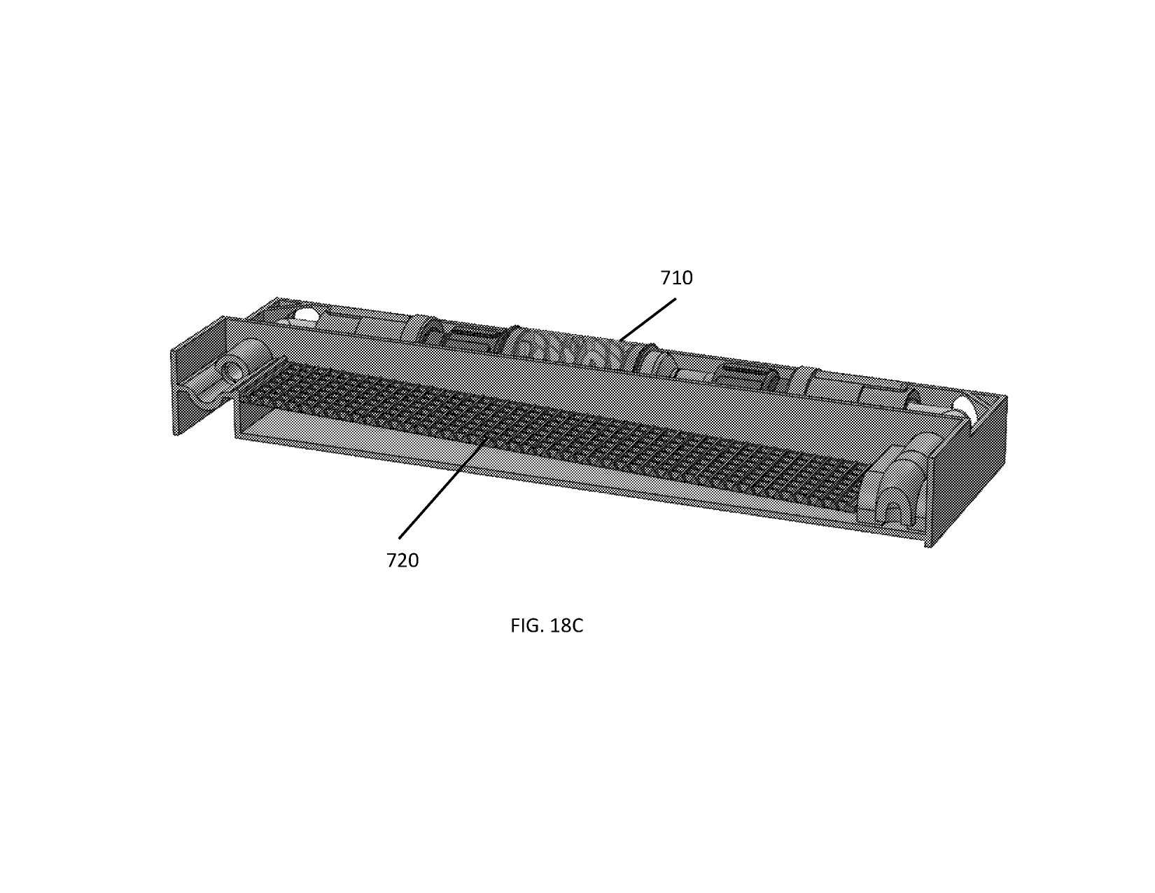

FIGS. 18A-18C show a multiwell plate system, generally designated by reference number 700, according to an exemplary embodiment of the present invention. The multiwell plate system 700 includes one or more rotational media pumps 710 (such as, for example, media pump 300, 400 or 500, or combinations of such pumps) arranged around one or more multiwell plates 720. As shown best in FIG. 18C, the one or more multiwell plates 720 are arranged in a titled manner so that one end of each multiwell plate 720 is higher than an opposite end, thereby facilitating cell transfer and distribution to multiple wells. The bottom of the multiwell plates 720 may include a porous membrane. One or more connections 730 are arranged between the one or more media pumps 710 and the multiwell plates 720. The one or more media pumps 710 may be actuated by rotation (e.g., by rollers arranged below the media pumps 710), thereby resulting in re-circulation of fluid from the lower ends of the multiwell plates 720 to the higher ends, after the fluid has flowed due to gravity down the multiwell plates 720.

Also, in terms of fluidic connections, two ports may be located on a single end of the device to facilitate medium exchange (in/out). This would be also advantageous from a usability perspective to simply ‘plug in’ the device into an additional unit that serves this function (medium source/destination).

In some embodiments, the floating tissue supports may be removed from the device, with tissue(s)/ cells/ organoids, etc. and directly processed for histology.

Although the present disclosure herein has been described with reference to particular embodiments, it is to be understood that these embodiments are merely illustrative of the principles and applications of the present disclosure.

It is intended that the specification and examples be considered as exemplary only, with a true scope of the present disclosure being indicated by the following claims.

Additionally, all of the disclosed features of a device may be transposed, alone or in combination, to a system or method and vice versa.

1. A perfusion vessel comprising:
a body extending between a first end and a second end and being configured to be rotated about an axis of rotation, the body including one or more helical conduits configured to receive a fluid and extending between the first end and the second end of the body along an exterior surface of the body, the body defining a reservoir configured to receive the fluid and extending between the first end and the second end of the body, and the body defining a first aperture and a second aperture each in communication with the one or more helical conduits and the reservoir and configured for passage of the fluid; and
optionally a support extending between a first end and a second end, the support being received within the reservoir of the body, the support including a plurality of wells configured to accommodate biological cells and a permeable membrane disposed below the plurality of wells;
wherein the helical conduit of the body is configured to facilitate movement of the fluid between the first aperture and the second aperture of the body during rotation of the body.

2. The perfusion vessel of claim 1, wherein the perfusion vessel is configured for culturing biological cells.

3. The perfusion vessel of claim 1, wherein the support is configured to maintain orientation relative to a longitudinal axis of the body during rotation of the body.

4. The perfusion vessel of claim 1, wherein the support includes a first wall and a second wall extending substantially perpendicular to the permeable membrane, the first wall extends to a first height, the second wall extends to a second height, and the first height of the first wall is greater than the second height of the second wall.

5. The perfusion vessel of claim 4, wherein the first wall is configured to contain a first column of the fluid when the fluid is received within the reservoir and the second wall is configured to contain a second column of the fluid when the fluid is received within the reservoir.

6. The perfusion vessel of claim 1, wherein the support comprises side walls having a concave shape.

7. The perfusion vessel of claim 1, further comprising a sheath disposed around the body, the sheath and the body forming a container that defines a chamber extending between a first end and a second end of the container.

8. The perfusion vessel of claim 7, wherein the container defines an opening at one or more of the first end and the second end of the container.

9. The perfusion vessel of claim 8, further comprising an endcap configured to releasably engage the opening of the sheath.

10. The perfusion vessel of claim 9, wherein one or more of the body and the endcap defines a flow port configured to direct the fluid into the reservoir.

11. The perfusion vessel of claim 10, wherein the flow port comprises one or more sloped or curved channels configured to facilitate movement of the fluid during rotation of the body.

12. The perfusion vessel of claim 7, wherein the sheath is at least partially gas permeable.

13. A perfusion vessel configured for culturing biological cells, the perfusion vessel comprising:
a body extending between a first end and a second end and being configured to be rotated about an axis of rotation, the body including one or more helical conduits configured to receive a fluid and extending between the first end and the second end of the body along an exterior surface of the body, the body defining a reservoir configured to receive the fluid and extending between the first end and the second end of the body, and the body defining a first aperture and a second aperture each in communication with the one or more helical conduits and the reservoir and configured for passage of the fluid, wherein the helical conduit of the body is configured to facilitate movement of the fluid between the first aperture and the second aperture of the body during rotation of the body.

14. The perfusion vessel of claim 13, wherein the vessel further comprises a support extending between a first end and a second end, the support being received within the reservoir of the body, the support including a plurality of wells configured to accommodate biological cells.

15. The perfusion vessel of claim 13, wherein the helical conduit is configured to facilitate movement of the fluid from the first aperture toward the second aperture during rotation of the body.

16. The perfusion vessel of claim 13, wherein the support is configured to remain orientation relative to a longitudinal axis of the body during rotation of the body.

17. The perfusion vessel of claim 13, wherein the support includes a first wall and a second wall extending substantially perpendicular to the permeable membrane, the first wall extends to a first height, the second wall extends to a second height, and the first height of the first wall is greater than the second height of the second wall.

18. A support comprising:
a base extending between a first end and a second end;
a platform extending across the base, the platform including one or more wells; and
a permeable membrane extending across the platform, wherein the permeable membrane is configured such that cellular material can adhere to a surface of the membrane.

19. The support of claim 18, wherein the permeable membrane is configured to establish an interstitial flow resistance and/or interstitial flow rate of the culture medium perfused through the permeable membrane.

20. The support of claim 19, wherein the permeable membrane extends across the platform at a position beneath the platform.
